SEQUENCE LISTING (96 sequences)

SEQ ID NO: 1 - protein, 913 aa, artificial sequence
  HBB-E6V-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 2 - protein, 913 aa, artificial sequence
  HBB-E6V-R1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 3 - protein, 1088 aa, artificial sequence
  HBB-T1-L1
MGDPKKKRKVIDYPYDVPDYAIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNMGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPQQVVAIASLPGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 4 - protein, 1094 aa, artificial sequence
  HBB-T1-R1
MGDPKKKRKVIDKETAAAKFERQHMDSIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 5 - DNA, 19 bp, artificial sequence
  HBB-in-out PCR-R6 degenerated primer
  misc_feature (12): k is g or t
  misc_feature (6): s is c or g
acgttsaccttkccccaca

SEQ ID NO: 6 - DNA, 21 bp, artificial sequence
  HBB-in-out PCR-F6
tcgttaccaagctgtgattcc

SEQ ID NO: 7 - DNA, 14 bp, artificial sequence
  TTR-V30M-L1 target sequence
ggccacattgatgg

SEQ ID NO: 8 - DNA, 14 bp, artificial sequence
  TTR-V30M-R1 target sequence
ctagatgctgtccg

SEQ ID NO: 9 - DNA, 41 bp, artificial sequence
  TTR-WT target sequence
cggccacattgatggcaggactgcctcggacagcatctaga

SEQ ID NO: 10 - DNA, 41 bp, artificial sequence
  TTR-V30M target sequence
tggccacattgatggcaggactgcctcggacagcatctaga

SEQ ID NO: 11 - DNA, 16 bp, artificial sequence
  HBB-E6V-L1 target sequence
ggagaagtctgccgtt

SEQ ID NO: 12 - DNA, 16 bp, artificial sequence
  HBB-E6V-R1 target sequence
ccacgttcaccttgcc

SEQ ID NO: 13 - DNA, 66 bp, artificial sequence
  HBB- WT target sequence
agatgcaccatggtgtctgtttgaggttgctagtgaacacagttgtgtcagaagcaaatgtaagca

SEQ ID NO: 14 - DNA, 46 bp, artificial sequence
  HBB -E6V target sequence
tggagaagtctgccgttactgccctgtggggcaaggtgaacgtgga

SEQ ID NO: 15 - DNA, 16 bp, artificial sequence
  HBB-T1-L1 target sequence
ctgacacaactgtgtt

SEQ ID NO: 16 - DNA, 16 bp, artificial sequence
  HBB-T1-R1 target sequence
gcaccatggtgtctgt

SEQ ID NO: 17 - DNA, 49 bp, artificial sequence
  HBB-WT TALEN target
tgcaccatggtgtctgtttgaggttgctagtgaacacagttgtgtcaga

SEQ ID NO: 18 - DNA, 66 bp, artificial sequence
  HBB-Mutant TALEN target
agatgtaccatggcgccggcttgaggttgctagtgaacacagttgtgtcagaagcaaatgtaagca

SEQ ID NO: 19 - DNA, 1472 bp, artificial sequence
  AAV (pCLS30649)
cctctagagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacttcatccacgttcaccttgccccacagggcagtaacggcagacttctcctcaggagtcagatgttttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaatggtacttgtgtgccaacgcattagctacgccagctacaactttctgatatgcagcctgaaccgggggcgtaaactcctttccaaaatggtgagcaagcacgcagacgaggacgttaccaagcagtctaaaattttctggatcaacatgcagcttgtcacaatgcagctctgaaagcgttgcaaaggtccctttcaggttgtccaggtgcgccaggccatcggaaaaggcccccaaaacctttttgccatgtgctttgacctttggattccccatgacagcatcgggcgtggagagatccccgaaagattcgaaaaacctttgggtccacgggtacaccacgagcagtcggccaagggcttcccctccaacctcgtcaacgttgacctttccccacaacgccgttaccgcacttttctcctccggcgtcaggtgtaccatggcggcggcttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgtaatatttcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 17 bp, artificial sequence
  TN1 left target sequence
tataataatccttaggc

SEQ ID NO: 21 - DNA, 17 bp, artificial sequence
  TN1 right target sequence
tttggcaaaacaggaac

SEQ ID NO: 22 - DNA, 17 bp, artificial sequence
  TN2  left target sequence
taggcctcgatgggtgg

SEQ ID NO: 23 - DNA, 17 bp, artificial sequence
  TN2  right target sequence
tctaattccccttttgg

SEQ ID NO: 24 - DNA, 17 bp, artificial sequence
  TN3 left target sequence
ttagaagctgctattga

SEQ ID NO: 25 - DNA, 17 bp, artificial sequence
  TN3 righttarget sequence
tacatccacactcacct

SEQ ID NO: 26 - DNA, 17 bp, artificial sequence
  TN4  left target sequence
tttgacaatgcaatcat

SEQ ID NO: 27 - DNA, 17 bp, artificial sequence
  TN4 right target sequence
ttgaatacgcttaacat

SEQ ID NO: 28 - DNA, 17 bp, artificial sequence
  TN5 left target sequence
ttacaggccatgcacag

SEQ ID NO: 29 - DNA, 17 bp, artificial sequence
  TN5 right target sequence
tcgatacttataattcg

SEQ ID NO: 30 - DNA, 17 bp, artificial sequence
  TN6 left target sequence
tcggaaggcgaagatgc

SEQ ID NO: 31 - DNA, 17 bp, artificial sequence
  TN6 right target sequence
tgcgggaagcaaactgc

SEQ ID NO: 32 - DNA, 17 bp, artificial sequence
  TN7 left target sequence
tcccgcttcggtactct

SEQ ID NO: 33 - DNA, 17 bp, artificial sequence
  TN7 right target sequence
tacaacctgttgtccag

SEQ ID NO: 34 - DNA, 17 bp, artificial sequence
  TN8  left target sequence
taggccacttaccgccc

SEQ ID NO: 35 - DNA, 17 bp, artificial sequence
  TN8 right target sequence
tcccgttgctgcggtga

SEQ ID NO: 36 - DNA, 17 bp, artificial sequence
  TN9 left target sequence
ttaataatttcttcttc

SEQ ID NO: 37 - DNA, 17 bp, artificial sequence
  TN9 right target sequence
tgaccaaaagaagagga

SEQ ID NO: 38 - DNA, 17 bp, artificial sequence
  TN10 left target sequence
tgcgggttaatacaaca

SEQ ID NO: 39 - DNA, 17 bp, artificial sequence
  TN10 right target sequence
tcccggacaacaaacac

SEQ ID NO: 40 - DNA, 17 bp, artificial sequence
  TN11 left target sequence
tccgagaagaaattcca

SEQ ID NO: 41 - DNA, 17 bp, artificial sequence
  TN11 right target sequence
ttcaacaagttgcctat

SEQ ID NO: 42 - DNA, 17 bp, artificial sequence
  TN12 left target sequence
tgaaagaccttaacttg

SEQ ID NO: 43 - DNA, 17 bp, artificial sequence
  TN12 right target sequence
ttgtcaaggacttgttt

SEQ ID NO: 44 - DNA, 17 bp, artificial sequence
  TN13 left target sequence
ttcactaccggcgctcg

SEQ ID NO: 45 - DNA, 17 bp, artificial sequence
  TN13 right target sequence
taccagcacataatttc

SEQ ID NO: 46 - DNA, 17 bp, artificial sequence
  TN14 left target sequence
tgagcattgcttttatg

SEQ ID NO: 47 - DNA, 17 bp, artificial sequence
  TN14 right target sequence
ttcatccggcaaaatgg

SEQ ID NO: 48 - DNA, 17 bp, artificial sequence
  TN15 left target sequence
ttctgatagaagatata

SEQ ID NO: 49 - DNA, 17 bp, artificial sequence
  TN15 right target sequence
ttgctgggaacaatggt

SEQ ID NO: 50 - DNA, 17 bp, artificial sequence
  TN16 left target sequence
tttataataatccttag

SEQ ID NO: 51 - DNA, 17 bp, artificial sequence
  TN16 right target sequence
tggcaaaacaggaacca

SEQ ID NO: 52 - DNA, 17 bp, artificial sequence
  TN17 left target sequence
taggcctcgatgggtgg

SEQ ID NO: 53 - DNA, 17 bp, artificial sequence
  TN17 right target sequence
ttctaattccccttttg

SEQ ID NO: 54 - DNA, 17 bp, artificial sequence
  TN18 left target sequence
ttagaagctgctattga

SEQ ID NO: 55 - DNA, 17 bp, artificial sequence
  TN18 right target sequence
tacatccacactcacct

SEQ ID NO: 56 - DNA, 17 bp, artificial sequence
  TN19 left target sequence
tgcttctgctatgttaa

SEQ ID NO: 57 - DNA, 17 bp, artificial sequence
  TN19 right target sequence
tggactgtaatcatcgc

SEQ ID NO: 58 - DNA, 17 bp, artificial sequence
  TN20 left target sequence
ttacaggccatgcacag

SEQ ID NO: 59 - DNA, 17 bp, artificial sequence
  TN20 right target sequence
tcgatacttataattcg

SEQ ID NO: 60 - DNA, 17 bp, artificial sequence
  TN21 left target sequence
tcggaaggcgaagatgc

SEQ ID NO: 61 - DNA, 17 bp, artificial sequence
  TN21 right target sequence
tgcgggaagcaaactgc

SEQ ID NO: 62 - DNA, 17 bp, artificial sequence
  TN22 left target sequence
tcccgcttcggtactct

SEQ ID NO: 63 - DNA, 17 bp, artificial sequence
  TN22 right target sequence
tacaacctgttgtccag

SEQ ID NO: 64 - DNA, 17 bp, artificial sequence
  TN23 left target sequence
ttaccgcccatcaacgc

SEQ ID NO: 65 - DNA, 17 bp, artificial sequence
  TN23 right target sequence
tgtagcggtcccgttgc

SEQ ID NO: 66 - DNA, 17 bp, artificial sequence
  TN24 left target sequence
ttaataatttcttcttc

SEQ ID NO: 67 - DNA, 17 bp, artificial sequence
  TN24 right target sequence
tgaccaaaagaagagga

SEQ ID NO: 68 - DNA, 17 bp, artificial sequence
  TN25 left target sequence
tgcgggttaatacaaca

SEQ ID NO: 69 - DNA, 17 bp, artificial sequence
  TN25 right target sequence
tcccggacaacaaacac

SEQ ID NO: 70 - DNA, 17 bp, artificial sequence
  TN26 left target sequence
tgataggcaacttgttg

SEQ ID NO: 71 - DNA, 17 bp, artificial sequence
  TN26 right target sequence
ttggaatcagcgacaag

SEQ ID NO: 72 - DNA, 17 bp, artificial sequence
  TN27 left target sequence
ttgaaagaccttaactt

SEQ ID NO: 73 - DNA, 17 bp, artificial sequence
  TN27 right target sequence
tgtcaaggacttgtttg

SEQ ID NO: 74 - DNA, 17 bp, artificial sequence
  TN28 left target sequence
tgaaagaccttaacttg

SEQ ID NO: 75 - DNA, 17 bp, artificial sequence
  TN28 right target sequence
ttgtcaaggacttgttt

SEQ ID NO: 76 - DNA, 17 bp, artificial sequence
  TN29 left target sequence
ttcactaccggcgctcg

SEQ ID NO: 77 - DNA, 17 bp, artificial sequence
  TN29 right target sequence
taccagcacataatttc

SEQ ID NO: 78 - DNA, 17 bp, artificial sequence
  TN30 left target sequence
tgagcattgcttttatg

SEQ ID NO: 79 - DNA, 17 bp, artificial sequence
  TN30 right target sequence
tatttcatccggcaaaa

SEQ ID NO: 80 - DNA, 17 bp, artificial sequence
  TN31 left target sequence
tttctgatagaagatat

SEQ ID NO: 81 - DNA, 17 bp, artificial sequence
  TN31 right target sequence
tgctgggaacaatggtg

SEQ ID NO: 82 - DNA, 49 bp, artificial sequence
  WT HBB targeted locus
tgtttgaggttgctagtgaacacagttgtgtcagaagcaaatgtaagca

SEQ ID NO: 83 - DNA, 49 bp, artificial sequence
  Template HBB-Mut2
ggcttgaggttgacagtgaacacagttgtgtcagaagcaaatgtaagca

SEQ ID NO: 84 - DNA, 49 bp, artificial sequence
  Template HBB-Mut3
ggcttgaggttgacagtgaacacagttgtgtcagaagcaagcgtaagca

SEQ ID NO: 85 - DNA, 2814 bp, artificial sequence
  pCLS31540 (TALEN HBB T2 Left)
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 86 - DNA, 17 bp, artificial sequence
  target TALEN HBB T2 Left
tgcttacatttgcttct

SEQ ID NO: 87 - DNA, 2814 bp, artificial sequence
  pCLS31541 (TALEN HBB2 T2 Right)
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 88 - DNA, 17 bp, artificial sequence
  target TALEN HBB T2 Right
tgtttgaggttgctagt

SEQ ID NO: 89 - DNA, 2745 bp, artificial sequence
  pCLS31548 (TALEN HBB T3 Left)
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccgagtggcagcggaagtggcggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 90 - DNA, 17 bp, artificial sequence
  target pCLS31548 (TALEN HBB T3 Left)
ttacatttgcttctgac

SEQ ID NO: 91 - DNA, 2745 bp, artificial sequence
  pCLS31549 (TALEN HBB T3 Right)
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccgagtggcagcggaagtggcggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 92 - DNA, 17 bp, artificial sequence
  target pCLS31549 TALEN HBB T3 Right
tgtttgaggttgctagt

SEQ ID NO: 93 - protein, 936 aa, artificial sequence
  TALEN HBB T2 Left
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 94 - protein, 936 aa, artificial sequence
  TALEN HBB T2 Right
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 95 - protein, 913 aa, artificial sequence
  TALEN HBB T3 Left
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 96 - protein, 913 aa, artificial sequence
  TALEN HBB T3 Right
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

(19) United States
(12) Patent Application Publication
(10) Pub. No.: US 2021/0017545 A1
(43) Pub. Date: Jan. 21, 2021

(54) TALE-NUCLEASES FOR ALLELE-SPECIFIC CODON MODIFICATION AND MULTIPLEXING

(71) Applicant: CELLECTIS, Paris (FR)

(72) Inventors: Alex BOYNE, Jersey City, NJ (US); Brian BUSSER, New York, NY (US); Philippe DUCHATEAU, Draveil (FR); Aymeric DUCLERT, ST MAUR DES FOSSES (FR)

(21) Appl. No.: 17/041,359

(22) PCT Filed: Mar. 29, 2019
(86) PCT No.: PCT/EP2019/058093

§ 371 Date: Sep. 24, 2020

Related U.S. Application Data
(60) Provisional application No. 62/649,871, filed on Mar. 29, 2018.

(30) Foreign Application Priority Data
Sep. 27, 2018 (DK) PA201870633

(51) Int. Cl. C12N 15/90; C12N 15/86; C07K 14/805; C12N 15/10; C12N 9/22

(52) U.S. Cl. CPC C12N 15/907; C12N 15/86; C07K 14/805; C12N 2750/14143; C12N 9/22; C07K 2319/81; C12N 15/102

(57) ABSTRACT

The present invention relates to the field of genome engineering (gene editing). More specifically the invention provides with allele specific TALE-nucleases and methods to operate allele specific gene repair by homologous recombination in primary cells, such as hematopoietic stem cells, blood cells and hepatocytes. These reagents and methods can be used for the genetic treatment of inherited disease, such as sickle cell disease betathalassemia.

### FIELD OF THE INVENTION

The present invention relates to the field of genome engineering (gene editing). More specifically the invention provides with allele specific TALE-nucleases and methods to operate allele specific gene repair by homologous recombination in primary cells, such as hematopoietic stem cells, blood cells and hepatocytes. These reagents and methods can be used for the genetic treatment of inherited disease, such as sickle cell disease, Beta thalassemia.

### BACKGROUND OF THE INVENTION

The past few years have seen the emergence of two major nuclease-based gene-editing platforms namely the transcription activator like effectors (TALE) and the clustered regularly interspaced short palindromic repeats (CRISPR).

Transcription activator-like effectors (TALEs) are site-specific DNA-binding proteins originating from the plant pathogen Xanthomonas sp. [23, 24]. The DNA-binding domain of TALEs are composed of an array of motifs of 33-35 amino acids repeats, which differ essentially by their residues 12 and 13 named RVDs (repeat variable diresidues). Critically, the base preference of a TALE repeat is substantially determined by these RVDs. In natural TALEs, the four most common RVDs NI, HD, NN and NG tend to specify bases A, C, G/A and T respectively. By following this RVD base-recognition specificity code, artificial TALE binding domains can be generated by assembly of selected RVDs to target specific desired DNA sequences, referred to as “target sequences”. So far, researchers have classically used TALE-nucleases heterodimeric architecture (commercially available under Cellectis Trademark TALEN®) based on the fusion of Fok1 catalytic head to C-terminal of the wild type protein AvrBs3. Fok1 catalytic head requires dimerization to be active, which requires that two TAL monomers facing each other on the two opposite DNA strands (right and left heterodimers) fused to Fok1 dimerize to recompose an active molecule [Christian et al. (2010) Targeting DNA double-strand breaks with TAL effector nucleases (2010) Genetics. 186(2):757-761]. TALE-nucleases can be designed to target almost any double stranded polynucleotide sequence. The only requirement is that the targeted sequences has to start with a thymine base (T₀) for an effective binding by the first RVDs of the protein located at the N-terminal domain of the TAL [Moscou, M. J. (2009) A Simple Cipher Governs DNA Recognition by TAL Effectors. Science. 326:1501]. This “T requirement” significantly drives the possibilities of targeting nucleotide sequence into the genome. However, this is not too limiting in terms of cleavage sites because TALE-nucleases architecture can be adjusted. For instance, fusion linkers between the TALE binding domain and Fok1 can be adapted to modify the spacer length between the right and left binding sites and also the number or RVDs can be modified.

TALEN-mediated genome editing has been demonstrated in diverse species and cell types, including human primary cells, hematopoietic stem cells and induced pluripotent stem cells. Studies have established TALE-nucleases as attractive reagents for genome editing that are somewhat easier to engineer than zinc-finger nucleases yet offer substantially higher targeting densities (up to tenfold) than systems based on CRISPR. Current TALE-nuclease architectures have turned out to constitute a very robust DNA targeting platform for therapeutic applications, such as for the production of allogeneic T-cells by gene inactivation. This has led to the first cancer treatment ever performed with gene-edited T-cells [Waseem Q. et al. (2017) Molecular remission of infant B-ALL after infusion of universal TALEN gene-edited CAR T cells. Science Translational Medicine. 9(374)].

Clustered regularly interspaced short palindromic repeat (CRISPR) is an essential component of nucleic-acid-based adaptive immune systems that are common in bacteria and archaea. In vitro reconstitution of the S. pyogenes type II CRISPR system has demonstrated that CRISPR RNA (crRNA) that is base-paired to trans-activating crRNA (tracrRNA), was acting as a RNA-guide to forms a two-RNA structure that directs Cas9 endonuclease to cleave DNA. This has opened the space to various RNA guided endonuclease systems broadly referred to as “CRISPR”. In such RNA-guided systems, the nuclease is directed to the genomic sequences that are complementary to the 20-nucleotide crRNA-guide sequence and followed by a PAM (protospacer-adjacent Motif) trinucleotide signature. At these sites, Cas9 (and more recently Cpf1) cuts both DNA strands with separate enzymatic domains, the HNH nuclease domain and the RuvC-like domain, to generate a double strand break (DSB). Based on these findings, several groups have engineered the protein and RNA components of the bacterial type II CRISR systems in mammalian cells, and demonstrated that Cas9 nucleases can be directed by short RNAs to induce targeted cleavage at diverse endogenous genomic loci in nearly all types of cells.

This system is particularly suited for multiplexing gene editing in cells where simultaneous introduction of multiple gRNAs in conjunction with the expression of Cas9 can be performed to target multiple loci in the same time.

Both genome editing technologies provide efficient and precise genetic modification by introducing a double-strand break (DSB) at a specific target sequence, followed by the generation of desired modifications during the subsequent DNA break repair. There are two major DNA repair mechanisms: the dominant but error-prone non-homologous end joining (NHEJ) pathway and the less-frequent but precise homologous recombination (HR) pathway. If the break is resolved via NHEJ, it can lead to gene disruption by introducing minor insertions and deletions. In contrast, if the break is resolved via HR in the presence of designed donor DNA, precise gene correction and targeted gene addition can be achieved.

Gene repair by homologous recombination offers hopeful perspectives in gene therapy as specific endonucleases can be used to genetically correct various severe inherited diseases of the blood, immune and nervous systems, including primary immunodeficiencies, leukodystrophies, thalassaemia, haemophilia and retinal dystrophy. These strategies exploit the combination of nucleases with improved vector technologies to deliver by homologous recombination functional copies of genes in which the inherited mutations have been corrected [for review see Naldini, L. (2015) Gene therapy returns to centre stage. Nature 256:351]. In some trials, genetic material is transferred into haematopoietic stem cells (HSCs) or T lymphocytes (T cells) ex-vivo prior to their engraftment into patients and in others hepatocytes in the liver or photoreceptors in the retina are targeted directly in-vivo.

To achieve gene repair, artificial nucleases and an exogenous DNA template bearing homology to the target site and comprising the new sequence must be delivered to the cell. The approach has great potential for use in ex vivo gene therapy because the targeted integration of an expression cassette into a preselected genomic ‘safe harbor or the in situ reconstitution of a mutant gene would ensure robust and predictable expression without the risk of insertional mutagenesis. Several hurdles must be overcome before these strategies can be fully exploited. This is because the efficiency of HDR-mediated genome editing remains low in most primary cell types of relevance to gene therapy, such as HSCs. In addition, it is challenging to achieve the safe and feasible clinical translation of cell-therapy products when having to rely on selection and extensive ex vivo amplification of a few edited cell clones. The cellular response to DNA DSBs varies according to cell type and cell cycle and growth status, and ranges from repair by the different pathways to differentiation or apoptosis. Overall, how the cell chooses between NHEJ and HDR is poorly understood.

Consequently, multiple applications have been found for targeted genome editing in experimental and preclinical models. However, translating these applications to the clinic, however, require thorough assessment of the off-target activity of the selected nuclease and optimization of the therapy.

TALE-nucleases involving Fok-1 under heterodimeric form produce sticky ends upon cleavage, which is favorable to relegation and repair under the HR pathway, whereas Cas9 in the CRISPR system produces blunt ends, which tend to make HR more challenging.

Thus, although CRISPR/Cas system appears to be advantageous compared to TALE-Nucleases, in terms of cost, and scalability for production and use for multiplex genome targeting. TALE-Nucleases, which are independently designed for each locus remain more specific and more reliably used to perform HR. Indeed, TALE-nucleases working as dimers, their cleavage site is generally determined by both their left and right target sequences amounting their target specificity up to 36 bp of DNA per cleavage site. By contrast, the specificity of Cas9 in the type II CRISPR system only depends on the

RNA-guided nuclease associated with the PAM sequence, which does not go beyond 20 pb upstream of the PAM, in which only the 12 base “seed-sequence” are really critical, whereas the remaining 8 bases (non-seed) and even the PAM sequence can allow mismatches.

Under these circumstances, TALE-nucleases appear to be more precise tools than CRISPR, when performing homologous recombination into large genomes, especially in the context of gene therapy, and this holds true even when multiple gene integrations are sought.

Hemoglobinopathies, in particular β-thalassemia and sickle cell anemia, are disease caused by hundreds of different mutations across the hemoglobin subunit beta (HBB) gene that cause severe life-long anemia. Currently allogeneic HSC transfer is the only curative therapy to these life threatening affections. Sickle cell disease (SCD) is more particularly caused by a missense mutation at codon 6 of HBB (A-to-T transversion). Depending on the patients, this disease may be mono-allelic or bi-allelic. At present, the only curative treatment of SCD is allogeneic hematopoietic stem cell (HSC) transplantation. 6-year disease-free survival of >90% has been reported for transplants from HLA-matched sibling donors. 5 However, in the United States, <14% of patients have a matched sibling donor. 6 Transplants with matched unrelated donors are limited by donor availability and immunologic barriers, such as graft rejection and graft-versus-host disease. Attempts to extend allogeneic transplant for SCD to alternative donor sources is an area of ongoing effort. 7 The SCD community has been cautious to embrace allogeneic HSC transplant in part given its short-term morbidity and mortality risks, though nonmyeloablative preparative regimens may help mitigate these risks. Given that the current clinical approach to SCD is largely reliant upon supportive care and hydroxyurea, the development of definitive therapies based on genetic manipulation of autologous HSCs would constitute a major advance. Gene therapy has long been proposed as a potential cure for SCD as permanent delivery of a corrective or antisickling gene cassette into long-term, repopulating HSCs could allow for the production of corrected red blood cells for the life of the patient. Clinical trials are on-going using lentiviral vectors. However, these gene addition strategies present the risk of insertional oncogenesis due to the random insertions of the lentiviral vectors into the genome. Correction of the sickle mutation by targeted nucleases followed by HDR in various cell types has been demonstrated, including reports of correction of induced pluripotent stem cells from both mice and humans [Hoban M. D. et al. (2016) Genetic treatment of a molecular disorder: gene therapy approaches to sickle cell disease. Blood. 127:839-848]. In addition, oligonucleotide-based gene therapy strategies, such as triplex-forming peptide nucleic acids which rely on HDR but not on the initial formation of a double-stranded break, have achieved low-frequency correction of the SCD mutation. Although these approaches offer the possibility to determine genome modification specificity on a clonal level, derivation of functional HSCs from pluripotent cells remains a great challenge. Recently, correction in human HSCs was reported. However, the rates of correction in long-term HSCs were well below levels necessary for therapeutic benefit. A similar finding of preferential utilization of NHEJ in HSCs (despite relatively robust HDR repair in unfractionated CD34⁺ hematopoietic stem and progenitor cells) has been observed in experiments attempting to correct the SCID-X1 mutation in human HSCs. A simple explanation of this observation may be that the HDR pathway is restricted to the S and G2 phases of the cell cycle when sister chromatids are available as donor repair template sequences. In contrast, HSCs, which are largely quiescent cells, rely mainly on NHEJ. [Genovese P., et al (2014) Targeted genome editing in human repopulating haematopoietic stem cells. Nature 510 (7504):235-240]

Similarly, familial transthyretin (TTR) amyloidosis is a autosomal genetic disease Each child of an affected individual (who is heterozygous for one TTR pathogenic variant) has a 50% chance of inheriting the TTR variant. Transthyretin (TTR) is a transport protein (Uniprot ref. #P02766) in the serum and cerebrospinal fluid that carries the thyroid hormone thyroxine (T4) and retinol-binding protein bound to retinol. This is how transthyretin gained its name: transports thyroxine and retinol. The liver secretes transthyretin into the blood, and the choroid plexus secretes TTR into the cerebrospinal fluid. The result of mutation in TTR a slowly progressive peripheral sensorimotor neuropathy and autonomic neuropathy as well as non-neuropathic changes of cardiomyopathy, nephropathy, vitreous opacities, and CNS amyloidosis. Point mutations within TTR are known to destabilize the tetramer composed of mutant and wild-type TTR subunits, facilitating more facile dissociation and/or misfolding and amyloidogenesis. Replacement of valine by methionine at position 30 (TTR V30M) is the mutation most commonly associated with familial amyloid polyneuropathy [Saraiva M. J. (1995) Transthyretin mutations in health and disease. Hum. Mutat. 5 (3): 191-6]. Only one copy of the defective gene is sufficient to cause the disorder. The liver secretes transthyretin into the blood, and the choroid plexus secretes TTR into the cerebrospinal fluid. Treatment of familial TTR amyloid disease has historically relied on liver transplantation as a crude form of gene therapy. Because TTR is primarily produced in the liver, replacement of a liver containing a mutant TTR gene with a normal gene is able to reduce the mutant TTR levels in the body to less than 5%. However liver transplantation is life threatening and has adverse consequences. Allele-specific gene repair would thus also offer a much safer alternative if nucleases were able to segregate alleles that need to be corrected without harming functional ones.

The present invention aims to overcome the current limitations presented above by providing a general method to improve gene correction into cells induced by specific design of TALE-nucleases, which is applicable both to gene therapy and multiplexing gene editing.

### BRIEF SUMMARY OF THE INVENTION

Genome editing using programmable nucleases such as meganucleases, transcription activator-like effector nucleases (TALEN®), megaTAL, zinc finger nucleases (ZFNs), and clustered regularly interspersed short palindromic repeats (CRISPR/Cas) is rapidly being applied to the treatment of genetic disease. Current strategies take advantage of the error-prone non-homologous end-joining (NHEJ) pathway to introduce small insertions or deletions (indels) in the target gene following repair of the double stranded break (DSB). There has been extensive study of programmable nucleases that aims to control their targeting specificity by mitigating the potential of recognizing off-target sites and the possibility of targeting particular alleles. The latter provides an opportunity to create nucleases that discriminate wild-type and mutant alleles to selectively inactivate the mutant allele in various genetic diseases that includes autosomal dominant diseases.

However, targeting a programmable nuclease to discriminate single nucleotide changes is a challenge as mismatching between the engineered protein (or guide RNA in the case of CRISPR/Cas) and target sequence can cause cleavage of the wild-type allele. Alternative genome editing approaches are needed to target particular alleles. For CRISPR-Cas, the requirement for the protospacer adjacent motif (PAM) immediately following the DNA target sequence can be exploited to target specific alleles. However, the necessity to utilize the PAM sequence for targeting limits the alleles available due to the strict sequence requirements on the PAM sequence (usually any nucleotide followed by two guanines, NGG).

On another hand, the DNA binding of transcription activator-like effectors (TALE) is mediated by a tandem array of 33 to 35 amino acid-long repeats with each of the individual repeated modules differing at the repeat variable di-residue (RVD) that recognizes a single base on the DNA. The RVD recognition code has been used to generate TALEs of custom-designed DNA binding specificities with the specificity of a TALE always preceded by the nucleotide thymidine (T) at repeat 0 (T₀).

In the present invention, the inventors have more particularly taken advantage of the functional requirement for a T₀ in TALEN® to design programmable nucleases that target particular alleles that contain a “T”.

By the general method of the present invention, the inventors have designed and produced TALE-nucleases that preferentially cleave alleles that contain T at the first position, such as one targeting the V30M allele of transthyretin (TTR) characteristic of transthyretin amyloidosis and another targeting the E6V allele of hemoglobin B (HBB) characteristic of sickle cell anemia.

In more specific aspects, the invention relies on the design of allele-specific TALE-nucleases, which target small nucleotide polymorphisms (SNP) in the mutant allele that comprises a T that serves as T₀ position for these TALE-nucleases. Allele-specific gene function can be modulated by fusing the TALE to a nuclease such as Fok1 or a monomeric meganuclease as non-limiting examples, a transcriptional activator such as vp64 (an engineered tetramer of herpes simplex VP16 transcriptional activator domain), the activation domain of p65 or the Epstein-Barr virus R transactivator (Rta) as non-limiting examples, or a transcriptional repressor such as the Kruppel-associated box (KRAB) or the mSin3 interaction domain (SID) as non-limiting examples.

Such allele-specific TALEs allow to discriminate mutated and wild type gene sequences, which is particularly useful in gene therapy to perform gene repair of pathological allelic forms. In particular, the invention provides combining such allele-specific TALE-nucleases with DNA template to correct the defective allele in which the wrong codon comprising T that serves as T₀ position for the TALE-nucleases is being removed or replaced upon homologous recombination. By doing so, the TALE-nuclease, cannot cleave again the repaired allele and progressively all defective alleles get repaired.

In this application, emphasis is given to methods for treating disease related to HBB gene mutations, such as sickle cell anemia and beta thalassemia, involving HSCs that are genetically modified ex-vivo following the teachings of the present invention. Such methods more particularly provide polynucleotide template sequences for homologous directed gene replacement (HDR) that comprise repaired HBB coding sequence preceded by a promoter region or 5′ UTR region, homologous to the wild type, that has been mutated, more particularly in the kozak sequences, to prevent re-cutting by the rare-cutting endonuclease being used for the integration of this polynucleotide template at the HBB locus. Examples of specific TALE-nucleases targeting the HBB promoter region according to the invention are also provided alone or in combination with the polynucleotide templates.

By pursuing the approach of integrating DNA template comprising codon that introduce mutations into the rare-cutting endonuclease target sequence, so that the said endonuclease does not recognize the modified locus upon recombination, the present invention provides a method for substituting codons genome-wide. The codons to be substitute can be homologous codons (i.e. without any impact on protein translation), stop codons or codons that will result into amino acid substitutions. In particular, the invention allows multiplexing codon changes since once recombination occurs, the TALE-nuclease cannot bind and cleave the modified locus anymore. The codon changes are thereby unlikely to revert and mutations can be stacked into cells genomes. The invention is particularly suited for replacing codons comprising a T by stop codons that will lock expression at selected locus.

The present invention actually greatly expands the allele-specific editing toolkit of programmable nucleases as actually over 90% of possible codons contain a T.

### BRIEF DESCRIPTION OF THE FIGURES AND TABLES

The patent or application file contains at least one drawing executed in color. Copies of this patent or patent application publication with color drawing(s) will be provided by the Office upon request and payment of the necessary fee.

FIG. 1: Schematic of TALEN® recognition of a nucleotide sequence. An array of TAL DNA binding domains that contain a 33-34 amino acid sequence that diverges at amino acids 12 and 13 (so-called repeat variable diresidue (RVD) is engineered to target a particular sequence of DNA. For DNA cleavage, each half of the non-specific Fok1 endonuclease is fused to the TALE array to create a TALEN® that cleaves the DNA between the RVDs. Alternative effector domains can be fused to the TALE such as activating or repressing proteins to manipulate gene activity in predictable ways.

FIG. 2: Allele-specific TALEN® according to the invention designed to target To as part of the codon to be substituted (A) Sequences of the WT and V30M alleles of TTR are shown in the upper part. TALEN® were designed to recognize the underlined sequences of the V3OM allele, with the codon replacement created in the V30M allele highlighted removing To. Genomic DNA from 293T cells that have integrated a wild-type (WT) copy or the V3OM version of TTR was isolated from cells transfected with RNA encoding a V30M targeting TALEN® and used in a T7 endonuclease 1 (T7E1) assay. T7E1 degradation products are marked with arrows. (B) Sequence of the WT and E6V alleles of HBB are shown in the upper part. TALEN® were designed to recognize the underlined sequences of the E6V allele, with the novel T created in the E6V allele highlighted. Genomic DNA from WT cells (Raji) or those that harbor the E6V sickle cell allele (SC-1) was isolated from cells transfected with RNA encoding an E6V targeting TALEN® and used in a T7 endonuclease 1 (T7E1) assay. T7E1 degradation products are marked. More details are provided in Example 1.

FIG. 3: Strategy to repair HBB allele using specifically designed HBB TALEN® (SEQ ID NO:1) and associated nucleic acid template comprising mutated target site. (A) Mutations in the TALE recognition sequence in the wild type WT HBB target site (SEQ ID NO:3) by replacement of a synonymous codon (GTC→GTA) to obtain functional HBB uncleavable site (SEQ ID NO: 4). (B) Diagram showing results of the Extrachromosomal assay detailed in Example 1, showing that cleavage by HBB-TALEN is abrogated on HBB uncleavable site.

FIG. 4: Strategy to repair HBB allele as per the present invention using HBB TALEN® (ex: HBB T1, T2 and T3) and associated polynucleotide template comprising specifically designed mutations in the target sequence of said TALEN® . The mutations are selected to prevent TALEN recutting of the repaired HBB locus upon integration of the polynucleotide template. They are also designed to concomitantly optimize kozak sequence upstream the HBB coding sequence. In both HBB-Mut2 and HBB-Mut3, the templates are mutated in the target sequence of HBB T2 R and HBB T3 R (SEQ ID NO:88 and SEQ ID NO:90 respectively) to remove the T0 initiating TALE-nuclease binding, upon integration of said template at the locus. (A) Alignment of sequences showing the mutations in the TALE recognition sequence relative to the wild type WT HBB target site. HBB-Mut depict TALEN target positions (underlined) and mutations described in Example 2 (also shown in FIG. 2). HBB-Mut2 and HBB-Mut3 depict TALEN target positions (underlined) and mutations described in Example 3 related to TALEN pair HBB T2 R and L (SEQ ID NO:94 and 93). (B) Same alignment of sequences as shown in (A), HBB-Mut2 and HBB-Mut3 depict TALEN target positions (underlined) and mutations described in Example 3 related to TALEN pair HBB T3 R and L (SEQ ID NO:96 and 95). (C) Diagram showing results of the Extrachromosomal assay detailed in Example 2, showing that the mutated target sites in the polynucleotide templates abrogate cleavage by HBB TALENs.

FIG. 5: Results of detection of integrated AAV repair template according to the invention. Modification of the HBB allele in HSCs was obtained by delivering an HBB TALEN with rAAV6 comprising a HBB repair template depicted as wild-type (WT), HR (containing the re-written HBB cDNA as per the present invention) or Indels (containing small insertions/deletions at the TALEN cleavage site).

FIG. 6: Modification of the HBB allele in HSCs by delivering a HBB TALEN® (SEQ ID NO:1) with rAAV6 delivering a HBB repair template that incorporates mutations that preclude template re-cutting by TALEN. (A) Preferred approach according to the invention involving a DNA template in which a synonymous codon is replaced in the HBB left target sequence. (B) Alternative approach involving the removal of the HBB right target sequence.(C) Time frame of transfection of the primary HSCs with the AAV vectors which are used as DNA templates.

FIG. 7: Results of PCR detection of integrated AAV repair template. Three biological samples were tested in duplicate: unmanipulated HSCs treated with rAAV6, mock-transfected HSCs treated with rAAV6 and HBB TALEN transfected HSCs treated with rAAV6. (A) 50 ng of genomic DNA isolated from treated HSCs was used in two separate 35-cycle PCR reactions, one that selectively amplifies the modified allele using in-out PCR and another that amplifies a genomic region outside of the HBB locus. (B) qPCR assay that selectively amplifies the modified allele versus the unmodified wild-type allele.

FIG. 8: Diagram showing results and comparison of allele frequencies in the modified HSCs determined by qPCR characterization of HBB modification. The qPCR assay show that more than 10% repair could be achieved in the transformed HSCs using repair template with proper mutations in the left TALEN® binding site which preclude cutting/re-cutting. By contrast, integration was very low using the approach involving right target removal.

FIG. 9: Modified HSCs according to the method of the present invention can differentiate into myeloid and erythroid lineages. Individual erythroid colonies (CFU-E) were picked, genomic DNA extracted and assessed for gene repair using in-out PCR. The experiments detailed in example 2 show that at least 3 out of 8 (more than 30%) individual erythroid clones were modified.

FIG. 10: Approach detailed in Example 4 used to design TALE-nuclease for stop codon insertions at the locus USP9Y exon3 without additional insertion of synonymous codons (TALEN TN1, TN2 and TN3). Squared codons are those intended to be substituted by stop codons. Underlined base pairs are mutated positions into the nucleotide TALE target sequences.

FIG. 11: Approach used in Example 4 to design TALE-nuclease for stop codon insertions at the locus SRY exon1 without additional insertion of synonymous codons (TALEN TN4, TN5, TN6, TN7 and TN8). Squared codons are those intended to be substituted by stop codons. Underlined base pairs are mutated positions into the nucleotide TALE target sequences..

FIG. 12: Approach used in Example 4 to design TALE-nuclease for stop codon insertions at the locus PCDH11Y_exon1 without additional insertion of synonymous codons (TALEN TN9, TN10, TN11, TN12, TN13, TN14 and TN15). Squared codons are those intended to be substituted by stop codons. Underlined base pairs are mutated positions into the nucleotide TALE target sequences.

FIG. 13: Approach detailed in Example 4 used to design TALE-nuclease for stop codon insertions at the locus USP9Y exon3 with additional insertion of synonymous codons (TALEN TN16, TN17 and TN18). Squared codons are those intended to be substituted by stop codons. Underlined base pairs are mutated positions into the nucleotide TALE target sequences.

FIG. 14: Approach detailed in Example 4 used to design TALE-nuclease for stop codon insertions at the locus USP9Y exon3 with additional insertion of synonymous codons (TALEN TN19, TN20, TN21, TN22 and TN23). Squared codons are those intended to be substituted by stop codons. Underlined base pairs are mutated positions into the nucleotide TALE target sequences.

FIG. 15: Approach detailed in Example 4 used to design TALE-nuclease for stop codon insertions at the locus USP9Y exon3 with additional insertion of synonymous codons (TALEN TN24, TN25, TN26, TN27, TN28, TN29, TN30 and TN31). Squared codons are those intended to be substituted by stop codons. Underlined base pairs are mutated positions into the nucleotide TALE target sequences.

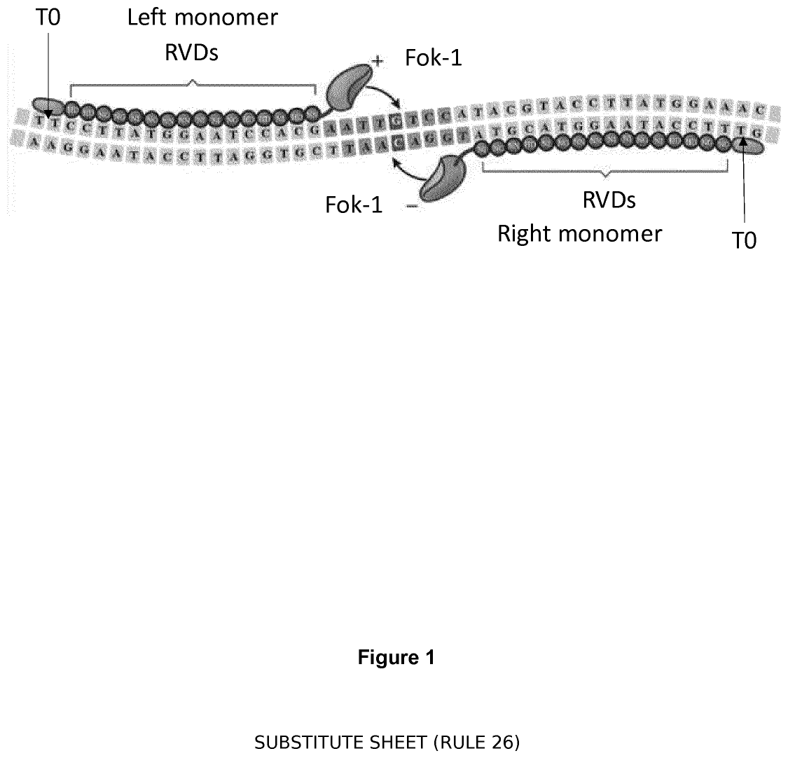

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols.154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

The present invention is drawn to methods for modifying one or several selected codon at a precise locus in a cell, wherein said method involves a TALE binding domain, preferably fused to a nuclease that binds a nucleotide sequence specific to said locus, referred to as “target sequence”. In general, said target sequence comprises at least an allele specific mutation, such as SNP (single nucleotide polymorphism) and said TALE is designed in such a way that, when this SNP is subsequently removed by gene repair, for instance upon HDR using a DNA template, said TALE does not recognize the repaired locus anymore. The SNP can be included, for instance in a codon that causes an amino acid substitution.

In general, this method comprises one or several of the following steps:

identifying a T (To) located at or at a distance less than 60 pb, preferably less than 30 pb of a selected codon to be modified at said endogenous locus,

identifying the polynucleotide target sequence starting from said T₀ in the 5′→3′ direction, which can be bound by a TALE binding domain. This can be done on a routine basis following the general rules previously established in the art (see for instance WO2011072246). Since the target sequence is likely to be modified during the following steps, it is referred as “initial target sequence”, also meaning that said target sequence can be allele-specific.

providing a nucleic acid template encompassing said target sequence that comprises a polynucleotide sequence at least 80%, preferably at least 90%, and generally more than 95% identical to the endogenous locus. In general, said template aims to correct gene defects by removing mutations. Thus, said nucleic acid template comprises
the replacement codon, referred to as “modified codon”, and optionally
at least one synonymous codon, which generally changes the target sequence without changing the amino acid sequence of the protein expressed at the locus.

According to a preferred embodiment of the invention, said modified codon and synonymous codon(s) are the only changes incorporated in the polynucleotide sequence of the nucleic acid template. In general, said modified codon and/or said optional synonymous codon(s) introduce mutation(s) into said polynucleotide target sequence.

providing a nucleic acid encoding a TALE-nuclease comprising a RVD sequence which has been designed to bind the initial target sequence, but which cannot bind the mutated target sequence once the modified codon has been inserted by homologous recombination,

introducing said nucleic acid template into the cell along with said nucleic acid encoding said TALE-nuclease.
As illustrated in the examples herein, the nucleic acid template is preferably included into an AAV vector. Said AAV vector can be transduced concomitantly or shortly after TALE-nuclease transfection, more preferably more than one hour after transfection of the nucleic acids expressing said TALE-nucleases. According to a preferred aspect of the invention said TALE-nuclease is expressed from transfected mRNA.

culturing the cells to allow expression of said TALE-nuclease, and subsequently, allele specific cleavage of the endogenous locus and insertion of the corrected codon at said locus by homologous recombination.

As shown in the Examples, the method of the present invention can be performed in different cell types, especially human cells, such as iPS, hepatocytes or primary hematopoietic stem cells.

As used herein, the term “hematopoietic stem cells” (or “HSC”) refer to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSC are CD34−. In addition, HSC also refer to long term repopulating HSC (LT-HSC) and short term repopulating HSC (ST-HSC). LT-HSC and ST-HSC are differentiated, based on functional potential and on cell surface marker expression. For example, in some embodiments, human HSC are a CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSC are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSC are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSC are less quiescent (i.e., more active) and more proliferative than LT-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSC can be used in any of the methods described herein. In some embodiments, ST-HSC are useful because they are highly proliferative and thus, can more quickly give rise to differentiated progeny.

By “nucleic acid template” is meant any nucleic acid that can be transfected into the cell and be accepted by cells gene repair enzymes as a template for homologous recombination. AAV vectors, especially AAV6, are particularly efficient DNA template that can transduced into cells under viral form.

According to a preferred aspect of the invention, said T₀ is included into said selected codon to be modified, and preferably removed upon insertion of the corrected codon at said locus by homologous recombination.

The method of the invention is particularly suited for performing unique or consecutive or simultaneous codon substitution(s) at one or several locus (loci). In this respect, the method of the present invention can be regarded as a method of directed mutagenesis, in which codon(s) located within a TALE-nuclease target sequence is(are) modified in such a way that said TALE-nuclease cannot specifically bind said target sequence once the codon has been modified. According to a preferred aspect, said selected codon is converted into a proteinogenic amino acid, so that amino acid substitution occurs at the protein level.

According to a preferred aspect, illustrated in Example 4 herein, selected codons can be converted or substituted into stop codons, such as TAG, TGA or TAA (modified codon). This can have a broad application genome-wide or within a gene network for multiplexing gene inactivation. Since the conversion of the selected codon into stop codon prevents retargeting of the allele-specific TALE-nuclease, the risk of reversion of the induced mutations gets lower.

According to another aspect of the present invention is a method for determining the “minimal peptidome” related to a cell function, or related to the survival a cell genome-wide in certain environmental conditions, said method comprising:
inactivating a cell at different loci using the method previously described;
culturing the cell over several generation to ensure maximal insertion of stop codons,
isolating the surviving cells,
determining which loci are mutated in the surviving cells and which are not; This method can be optionally developed by additional steps, such as
determining those loci that cannot be mutated alone or in combination by comparing the results obtained with different clones of surviving cells.

This method is particularly useful to study regulatory pathways and determine the genes, the expression of which is essential for a cell to survive in given environmental conditions. This is useful for instance to develop models for synthetic biology.

The present method can also be regarded as a method for mutating a cell line at different loci, wherein said method comprises at least one of the following steps:
identifying a T (T₀) located at or at a distance less than 60 pb, preferably less than 30 pb of a selected codon to be corrected at said endogenous locus;
identifying target sequence starting from said T0 in the 5′→3′ direction;
providing nucleic acid templates homologous to said endogenous locus, encompassing said target sequences and comprising stop or modified codon(s), and optionally at synonymous codon(s) for insertion by homologous recombination at the different specific loci upon cleavage by said TALE-nucleases, wherein said corrected codon and said optional synonymous codon(s) introduce mutation(s) into said polynucleotide target sequence,
providing nucleic acids encoding TALE-nucleases comprising RVD sequences which have been designed to bind the initial target sequences but which cannot bind said mutated target sequences when the stop or modified codons have been inserted by homologous recombination,
introducing into the cell said nucleic acid templates comprising said stop or modified codons along with the nucleic acids encoding said TALE-nucleases ;
culturing the cells to allow expression of said TALE-nucleases and the insertion of said stop codons at the different loci;
selecting the cells that have the stop codons inserted in their genomes at these loci.

According to another aspect of the present invention said selected codon can be converted into a synonymous codon (modified codon) for the purpose of recoding a gene or a entire genome.

In order to help discrimination by the TALE between the initial target sequence and that inserted by homologous recombination, from 2 to 5 synonymous codons, preferably from 2 to 3, can be introduced into the target polynucleotide sequence borne by the nucleic acid template.

The TALE-nuclease that are used according to the present invention is preferably a heterodimer member that has to dimerize with a second TALE-monomer, such as a TALE-fok1 monomer. According to preferred embodiments as illustrated herein, the selected codon is located in the spacer sequence—i.e. between the binding sequences of the first and second TALE monomers.

As evidenced in the experimental part of the present application, the present invention discloses specific TALE-nucleases intervening at different loci for allele specific gene correction of TTR and HBB.

In particular, the invention is drawn to allele specific TALE-nucleases useful for treating sickle cell disease directed to E6V mutated form of HBB, and for treating transthyretin as being directed to V3OM mutated form of TTR.

An example of TALE-nuclease useful for correcting E6V mutation is the HBB-E6V-L1 TALEN described herein, characterized in that it comprises the following RVD sequence: NN-NN-NI-NN-NI-NI-NN-NG-HD-NG-NN-HD-HD-NN-NG-NG.

Said TALE-nuclease comprises an amino acid sequence sharing identity with SEQ ID NO:3. and is preferably used with another TALEN monomer, such as HBB-E6V-R1 (SEQ ID NO:4).

Other examples of sequence specific reagents useful for modifying and repairing HBB locus are HBB-T1-L1, HBB-T1-R1, HBB-T2-R, HBB-T2-L, HBB-T3-L, and HBB-T3-R TALE-Nucleases referred to in Table 1, which uses are more particularly described in Examples 2 and 3.

The above TALE-nucleases are useful in therapy, such as for treating sickle cell anemia and beta-thalassemia. One such method of treatment comprises the steps of transfecting HSCs with the above TALE-Nuclease, preferably the HBB-E6V-L1 TALEN comprising the polypeptide sequence SEQ ID NO:3, preferably along with a nucleic acid template comprising wild type HBB-WT TALEN target of SEQ ID NO:17, such as a AAV vector.

According to some embodiments, the method for allele-specific codon modification at a locus in a cell, can be practiced by performing one or several of the following steps:

a) introducing into a cell a rare-cutting endonuclease that has been previously designed to bind and cleave a specific target sequence into an endogenous locus;

b) transfecting said cell with a polynucleotide template comprising said specific target sequence, wherein said target sequence has been mutated.

In general, said mutated target sequence, which has been included into said polynucleotide template:
is at least 80% identical to the target sequence at said endogenous locus;
is not cleavable anymore by said rare cutting endonuclease, and
said mutation does not impair the transcription of the endogenous locus upon integration of said polynucleotide template at said endogenous locus.

c) inducing cleavage by the rare-cutting endonuclease of said endogenous locus to integrate said polynucleotide template at said locus.

Step c) of inducing cleavage is generally obtained by culturing the cells in appropriate conditions to have an active cell cycle favorable to genetic recombination and repair mechanisms.

The mutation introduced into the target sequence comprised in the polynucleotide template may have an effect on the endogenous locus coding sequence. When it is introduced in the coding sequence, the mutation can convert a codon into a synonymous codon or a codon specifying a different amino acid.

When a synonymous codon is introduced, the mutation has the unique effect to make the target sequence uncleavable by the rare-cutting endonuclease.

Alternatively, when the mutation encodes a different amino acid, this can improve the expression of the (exogenous) coding sequence or even improve the functionality of the protein encoded by said endogenous locus, in the same time as preventing re-cutting of the sequence at the endogenous locus.

According to a preferred embodiment the mutated codon introduces a mutation that both makes the target sequence uncleavable and repairs a genetic defect, especially a genetic defect causing beta thalassemia, sickle cell anemia or TTR disease.

As per an embodiment of the invention, said mutation(s) introduced into the target sequence on said polynucleotide template are located in the 5′UTR region of the gene present at the endogenous locus, especially into the Kozak sequence (see for instance example 3), preferably in view of optimizing said Kozak sequences.

Kozak sequences are well known sequences that occur on eukaryotic mRNA playing a major role in the initiation of the translation process as described by Kozak, M. [Point mutations define a sequence flanking the AUG initiator codon that modulates translation by eukaryotic ribosomes (1986) Cell. 44(2):283-92]. Such sequences correspond generally to the consensus (gcc)gccRccAUGG, where
a lower-case letter denotes the most common base at a position where the base can nevertheless vary;
upper-case letters indicate highly conserved bases,

Preferably, the ‘AUGG’ sequence is constant and (gcc) is optional.

Interestingly, the mutations introduced by the inventors have been found to increase the amount of mRNA when the coding sequence was integrated at the locus.

Stability of the mRNA may also be sought the mutations as per the present invention into stabilizing cis-elements and PolyA sequences.

As previously explained, the cell is preferably a hematopoietic stem cell or a blood cell, preferably erythrocyte.

According to preferred embodiments, the endonuclease used in the method of the present invention is a fusion of a binding domain with Fok1, such as ZFN, TALE-Nuclease, more preferably said endonuclease is the fusion of a nuclease with TALE binding domain, such as a TALE-nuclease or Mega-TALE.

According to preferred embodiments, the endonuclease used in the method of the present invention is a RNA-guided endonuclease, such as CRISPR. Indeed, following the invention, RNA-guides can be design to hybridize a target sequence, wherein a polynucleotide template comprising said target sequence can be mutated making it uncleavable by the nuclease upon integration of said polynucleotide template at the endogenous locus by homologous recombination or NHEJ.

According to the present invention, TALE-nucleases (or Mega-TALE) are preferred endonucleases due to the possibility of removing the To recognized by the TALE binding domain from said target sequence to make the polynucleotide template uncleavable by the TALE-nuclease when it is integrated at the endogenous locus by homologous recombination or NHEJ.

According to preferred embodiments of the present invention, said polynucleotide template is comprised into an AAV vector, preferably an AAV6 vector. Such vectors are particularly suited to perform integration by homologous recombination directed by rare-cutting endonucleases as described for instance by Sather, B. D. et al. [Efficient modification of CCR5 in primary human hematopoietic cells using a megaTAL nuclease and AAV donor template (2015) Science translational medicine, 7(307), 307ra156].

According to another embodiment, said polynucleotide template can be an oligonucleotide, harboring microhomologies or not, for an insertion by NHEJ repair mechanism at the cleaved locus.

In some embodiments, methods of non-viral delivery of the polynucleotide template can be used such as electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

In some embodiments, electroporation steps can be used to transfect cells. In some embodiments, these steps are typically performed in closed chambers comprising parallel plate electrodes producing a pulse electric field between said parallel plate electrodes greater than 100 volts/cm and less than 5,000 volts/cm, substantially uniform throughout the treatment volume such as described in WO 2004/083379, which is incorporated by reference, especially from page 23, line 25 to page 29, line 11. One such electroporation chamber preferably has a geometric factor (cm-1) defined by the quotient of the electrode gap squared (cm2) divided by the chamber volume (cm3), wherein the geometric factor is less than or equal to 0.1 cm-1, wherein the suspension of the cells and the sequence specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

The nucleic acid template sequence may also be an oligonucleotide or more preferably a single strand oligonucleotide (ssODN) and be used for gene correction of the HBB mutation in the endogenous sequence. The oligonucleotide or ssODN may be may be electroporated into the cell, or may be introduced via other methods known in the art.

The method of the present invention has been particularly designed for the treatment of sickle cell disease and beta-thalassemia, by gene therapy, more particularly by integrating corrected polynucleotide sequences at the endogenous HBB locus using the endonucleases and template polynucleotides described herein.

According to a preferred embodiment, said rare-cutting endonuclease, which is preferably the TALE-nuclease HBB-E6V as suggested in the examples, binds a target sequence into HBB, such as SEQ ID NO:11, wherein the polynucleotide template comprises SEQ ID NO:13 (mutated target sequence).

According to a preferred embodiment, said rare-cutting endonuclease, which is preferably the TALE-nuclease HBB-T1 as suggested in the examples, binds a target sequence into HBB, such as SEQ ID NO:13, wherein the polynucleotide template comprises SEQ ID NO:14 (mutated target sequence).

According to a preferred embodiment, the invention provides with rare-cutting endonucleases, which are preferably the TALE-nucleases HBB-E6V as referred to in example 2, which bind a target sequence into HBB, such as SEQ ID NO:11, wherein the polynucleotide template comprises SEQ ID NO:13 (mutated target sequence).

According to a preferred embodiment, the invention provides with rare-cutting endonucleases, which are preferably TALE-nucleases HBB-T1-L1, HBB-T1-R1, HBB-T2-L HBB-T2-R, HBB-T3-L and HBB-T3-R referred to in Example 3, which bind a target sequence into HBB, such as SEQ ID NO:17, especially a target sequence selected from SEQ ID NO:15, SEQ ID NO:16, SEQ ID NO:86, SEQ ID NO:88, SEQ ID NO:90 and SEQ

ID NO:92, while providing a polynucleotide template comprising any of the sequence SEQ ID NO:18, SEQ ID NO:83 or SEQ ID NO:84.

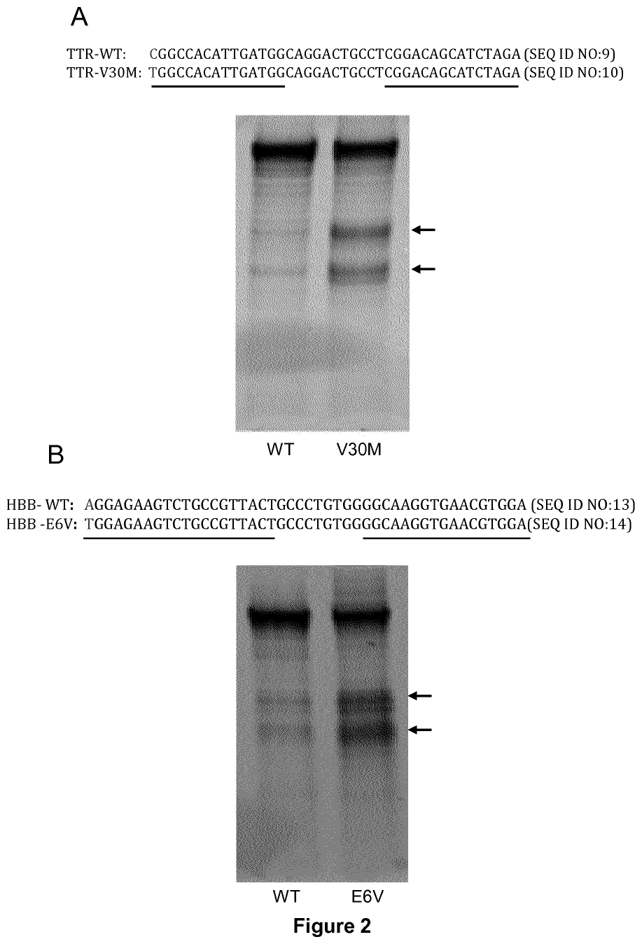

According to another embodiment shown in example 1 and FIG. 2A, the invention provides rare-cutting endonucleases that bind a target sequence into TTR gene (responsible for TTR amyloid disease), such as SEQ ID NO:10, which is preferably the TALE-nuclease TTR-V30M, while providing the polynucleotide template comprises SEQ ID NO:9 as mutated target sequence.

The invention also provides with kits for allele-specific codon modification at a locus in a cell, wherein said kit comprising a rare-cutting nuclease and its related polynucleotide template as previously described. Such kits typically comprise at least:
a polynucleotide encoding a rare-cutting endonuclease that has been designed to bind and cleave a specific target sequence into an endogenous locus;
a polynucleotide template comprising said specific target sequence, which has been mutated,
   wherein said mutated target sequence in said polynucleotide template:
      is at least 80% identical to the target sequence at said endogenous locus;
      is not cleavable by said rare cutting endonuclease, and
      said modified sequence does not impair the transcription of the endogenous locus upon integration of said polynucleotide template at said endogenous locus.

The invention also pertains to the engineered cell obtainable by the method previously described. Such cells are generally characterized in that it has been transfected with, and thus may comprise:
a rare-cutting endonuclease or a polynucleotide encoding thereof, that has been designed to bind and cleave a specific target sequence into an endogenous locus;
a polynucleotide template comprising said specific target sequence, which has been mutated,

wherein said mutated target sequence in said polynucleotide template:
is at least 80% identical to the target sequence at said endogenous locus;
is not cleavable by said rare cutting endonuclease, and
said modified sequence does not impair the transcription of the endogenous locus upon integration of said polynucleotide template at said endogenous locus.

Such engineered cell can comprise a polynucleotide sequence selected from HBB-mut1 (SEQ ID NO:18), HBB-mut2 (SEQ ID NO:83) or HBB-mut3 (SEQ ID NO:84) integrated at its HBB endogenous locus as illustrated in the experimental section herein.

In general, the genetic correction of the cells is performed ex-vivo and the treated cells are transplanted back to the patient suffering sickle cell disease or beta-thalassemia.

Example of rare cutting-endonucleases useful for correcting V3OM mutated form of TTR are also provided, especially the TALE-nuclease TTR-V30M-L1, with respect to the treatment of another inherited disease: familial Transthyretin.

TTR-V30M-L1 is characterized in that it comprises the following RVD sequence: NN-NN-HD-HD-NI-HD-NI-NG-NG-NN-NI-NG-NN-NG

Said TALE-nuclease comprises an amino acid sequence sharing identity with SEQ ID NO:2. and is preferably used with another TALEN monomer, such as TTR-V30M-R1 (SEQ ID NO:1). These TALE-nucleases are useful for therapy, such as for treating familial Transthyretin especially amyloid polyneuropathy. One such method of treatment comprises the steps of transfecting hepatocytes with the above TALE-Nuclease, preferably the TTR-V30M-L1 TALEN comprising the polypeptide sequence SEQ ID NO:2, preferably along with a nucleic acid template comprising wild type TTR WT target of SEQ ID NO:13, such as a AAV vector. In general, the treated cells are transplanted back to the patient suffering familial Transthyretin.

As further evidenced in the experimental art of the present disclosure, the present method bring into play allele specific TALE-nuclease that go along with specifically designed nucleic acid template(s). Both elements are inter-dependent, since the TALE-nuclease has to discriminate the target sequence borne by the nucleic acid template.

The invention thus relies on a kit for allele-specific codon modification at a locus in a cell, said kit comprising at least:
a nucleic acid template comprising a TALE target sequence from an endogenous locus that has been mutated by the insertion of a modified codon , and
a nucleic acid encoding a TALE-nuclease that has been designed such that the TALE nuclease that bind the endogenous target sequence does not recognize said mutated target sequence comprising said modified codon, in particular when said modified codon is inserted at said locus by homologous recombination.

Such kits are useful for therapy, such as gene therapy, and especially for the ex-vivo gene correction of blood cells. It preferentially comprises a TALE-nucleases as described herein, especially for the treatment of genetic disorders, such as TTR, beta-thalassemia and sickle cell anemia.

The present invention further relates to the TALE-nucleases generated as part of the experiments performed into PCDH11Yex1, SRY_ex1 and PCDH11Y_ex1 loci, characterized in that said TALE-nucleases comprise one RVD sequence selected from those listed into Tables 2 and 3.

The present invention further relates to modified cells or cell lines obtainable by any of the methods disclosed herein, especially in view of practicing cell transplantation into patients in need thereof.

The genetically modified cells can be administered either alone, or as a pharmaceutical composition in combination with diluents and/or with other components. In some embodiments, pharmaceutical compositions can comprise genetically modified HSC or iPS cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g. aluminum hydroxide); and preservatives. In some embodiments, compositions are formulated for intravenous administration.

In one embodiment, the invention provides a cryopreserved pharmaceutical composition comprising: (a) a viable composition of genetically modified HSC or iPS cells (b) an amount of cryopreservative sufficient for the cryopreservation of the HSC or iPS cells; and (c) a pharmaceutically acceptable carrier.

As used herein, “cryopreservation” refers to the preservation of cells by cooling to low sub-zero temperatures, such as (typically) 77 K or −196° C. (the boiling point of liquid nitrogen). At these low temperatures, any biological activity, including the biochemical reactions that would lead to cell death, is effectively stopped. Cryoprotective agents are often used at sub-zero temperatures to preserve the cells from damage due to freezing at low temperatures or warming to room temperature.

In some embodiments, the injurious effects associated with freezing can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO), glycerol, polyvinylpyrrolidine, polyethylene glycol, albumin, dextran, sucrose, ethylene glycol, i-erythritol, D-Sorbitol, D-mannitol, D-sorbitol, i-inositol, D-lactose, choline chloride, amino acids, methanol, acetamide, glycerol monoacetate, and inorganic salts. In a preferred embodiment, DMSO is used, a liquid which is nontoxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After the addition of DMSO, cells should be kept at 0-4° C. until freezing, since DMSO concentrations of about 1% are toxic at temperatures above 4° C.

Considerations and procedures for the manipulation, cryopreservation, and long-term storage of HSC, particularly from bone marrow or peripheral blood can be found, for example, in the following references, incorporated by reference herein: Gorin, N. C., 1986, Clinics In Haematology 15(1):19-48; Bone-Marrow Conservation, Culture and Transplantation, Proceedings of a Panel, Moscow, Jul. 22-26, 1968, International Atomic Energy Agency, Vienna, pp. 107-186.

Other methods of cryopreservation of viable cells, or modifications thereof, are available and envisioned for use (e.g., cold metal-minor techniques; Livesey, S. A. and Linner, J. G., 1987, Nature 327:255; Linner, J. G., et al., 1986, J. Histochem. Cytochem. 34(9):1123-1135; U.S. Pat. Nos. 4,199,022, 3,753,357, and 4,559,298 and all of these are incorporated hereby reference in their entirety.

After removal of the cryoprotective agent, cell count (e.g., by use of a hemocytometer) and viability testing (e.g., by trypan blue exclusion; Kuchler, R. J. 1977, Biochemical Methods in Cell Culture and Virology, Dowden, Hutchinson & Ross, Stroudsburg, Pa., pp. 18-19; 1964, Methods in Medical Research, Eisen, H. N., et al., eds., Vol. 10, Year Book Medical Publishers, Inc., Chicago, pp. 39-47) can be done to confirm cell survival.

The invention also pertains to therapeutic compositions comprising an effective amount of the engineered cells, or populations thereof, as described herein and illustrated in the experimental section, for their use as a medicament.

An “effective amount” or “therapeutically effective amount” refers to that amount of a composition described herein which, when administered to a subject (e.g., human), is sufficient to aid in treating a disease. The amount of a composition that constitutes a “therapeutically effective amount” will vary depending on the cell preparations, the condition and its severity, the manner of administration, and the age of the subject to be treated, but can be determined routinely by one of ordinary skill in the art having regard to his own knowledge and to this disclosure. When referring to an individual active ingredient or composition, administered alone, a therapeutically effective dose refers to that ingredient or composition alone. When referring to a combination, a therapeutically effective dose refers to combined amounts of the active ingredients, compositions or both that result in the therapeutic effect, whether administered serially, concurrently or simultaneously.

Other definitions:
Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “DNA target”, “DNA target sequence”, “target DNA sequence”, “nucleic acid target sequence”, “target sequence” , is intended a polynucleotide sequence which can be bound by the TALE DNA binding domain that is included in the proteins of the present invention. It refers to a specific DNA location, preferably a genomic location in a cell, but also a portion of genetic material that can exist independently to the main body of genetic material such as plasmids, episomes, virus, transposons or in organelles such as mitochondria or chloroplasts as non-limiting examples. The nucleic acid target sequence is defined by the 5′ to 3′ sequence of one strand of said target, as indicated for SEQ ID NO: 83 to 89 in table 3 as a non-limiting example. Generally, the DNA target is adjacent or in the proximity of the locus to be processed either upstream (5′ location) or downstream (3′ location). In a preferred embodiment, the target sequences and the proteins are designed in order to have said locus to be processed located between two such target sequences. Depending on the catalytic domains of the proteins, the target sequences may be distant from 5 to 50 bases (bp), preferably from 10 to 40 bp, more preferably from 15 to 30, even more preferably from 15 to 25 bp. These later distances define the spacer referred to in the description and the examples. It can also define the distance between the target sequence and the nucleic acid sequence being processed by the catalytic domain on the same molecule.
By “ delivery vector” or “ delivery vectors” is intended any delivery vector which can be used in the present invention to put into cell contact (i.e “contacting”) or deliver inside cells or subcellular compartments agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, peptides developed by Diatos. In these cases, delivery vectors are molecule carriers. By “delivery vector” or “delivery vectors” is also intended delivery methods to perform transfection.

The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors. A vector according to the present invention comprises, but is not limited to, a YAC (yeast artificial chromosome), a BAC (bacterial artificial), a baculovirus vector, a phage, a phagemid, a cosmid, a viral vector, a plasmid, a RNA vector or a linear or circular DNA or RNA molecule which may consist of chromosomal, non chromosomal, semi-synthetic or synthetic DNA. In general, expression vectors of utility in recombinant DNA techniques are often in the form of “plasmids” which refer generally to circular double stranded DNA loops which, in their vector form are not bound to the chromosome. Large numbers of suitable vectors are known to those of skill in the art. Vectors can comprise selectable markers, for example:

neomycin phosphotransferase, histidinol dehydrogenase, dihydrofolate reductase, hygro-mycin phosphotransferase, herpes simplex virus thymidine kinase, adenosine deaminase, glutamine synthetase, and hypoxanthine-guanine phosphoribosyl transferase for eukaryotic cell culture; TRP1 for S. cerevisiae; tetracyclin, rifampicin or ampicillin resistance in E. coli. Preferably said vectors are expression vectors, wherein a sequence encoding a polypeptide of interest is placed under control of appropriate transcriptional and translational control elements to permit production or synthesis of said polypeptide. Therefore, said polynucleotide is comprised in an expression cassette. More particularly, the vector comprises a replication origin, a promoter operatively linked to said encoding polynucleotide, a ribosome binding site, a RNA-splicing site (when genomic DNA is used), a polyadenylation site and a transcription termination site. It also can comprise an enhancer or silencer elements. Selection of the promoter will depend upon the cell in which the polypeptide is expressed. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-β-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes. Delivery vectors and vectors can be associated or combined with any cellular permeabilization techniques such as sonoporation or electroporation or derivatives of these techniques.
Viral vectors include retrovirus, adenovirus, parvovirus (e.g. adenoassociated viruses), coronavirus, negative strand RNA viruses such as orthomyxovirus (e.g., influenza virus), rhabdovirus (e. g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By cell or cells is intended any prokaryotic or eukaryotic living cells, cell lines derived from these organisms for in vitro cultures, primary cells from animal or plant origin.
By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines. These cells thus represent a more valuable model to the in vivo state they refer to.
In the frame of the present invention, the expression “double-strand break-induced mutagenesis” (DSB-induced mutagenesis) refers to a mutagenesis event consecutive to an NHEJ event following an endonuclease-induced DSB, leading to insertion/deletion at the cleavage site of an endonuclease.
By “gene” is meant the basic unit of heredity, consisting of a segment of DNA arranged in a linear manner along a chromosome, which codes for a specific protein or segment of protein. A gene typically includes a promoter, a 5′ untranslated region, one or more coding sequences (exons), optionally introns, a 3′ untranslated region. The gene may further comprise a terminator, enhancers and/or silencers.
As used herein, the term “locus” is the specific physical location of a DNA sequence (e.g. of a gene) on a chromosome. The term “locus” usually refers to the specific physical location of a polypeptide or chimeric protein's nucleic target sequence on a chromosome. Such a locus can comprise a target sequence that is recognized and/or cleaved by a polypeptide or a chimeric protein according to the invention. It is understood that the locus of interest of the present invention can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria or chloroplasts as non-limiting examples.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. Unless otherwise stated, the present invention encompasses polypeptides and polynucleotides sharing at least 70%, generally at least 80%, more generally at least 85%, preferably at least 90%, more preferably at least 95% and even more preferably at least 97% with those described herein.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

Below are summarized, without being exhaustive, certain embodiments of the present invention:
1) An Allele specific method for modifying a selected codon at a precise locus in a cell, wherein said method comprises the following steps:
   i) identifying a T (T₀) located at or at a distance less than 60 pb, preferably less than 30 pb of a selected codon to be modified at said endogenous locus,
   ii) identifying a initial polynucleotide target sequence starting from said To in the 5′→3′ direction, which can be bound by a TALE binding domain,
   iii) providing a nucleic acid template encompassing said target sequence, which is at least 80%, preferably at least 90%, identical to the endogenous locus, said template comprising:
      the modified codon, and optionally
      at least one synonymous codon,
   wherein said modified codon and/or said optional synonymous codon(s) introduce mutation(s) into said polynucleotide target sequence,
   iv) providing a nucleic acid encoding a TALE-nuclease comprising a RVD sequence which has been designed to bind the initial target sequence but which cannot bind the mutated target sequence when the modified codon has been inserted by homologous recombination,
   iv) introducing said nucleic acid template into the cell along with said nucleic acid encoding said TALE-nuclease,
   v) culturing the cells to allow expression of said TALE-nuclease, allele specific cleavage of the endogenous locus and insertion of the corrected codon at said locus by homologous recombination.
2) Method according to item 1, wherein said selected codon is converted into a stop codon TAG, TGA or TAA.
3) Method according to item 1, wherein said selected codon is converted into a synonymous codon.
4) Method according to item 1, wherein said selected codon is converted into one coding for a different amino acid, preferably a proteinogenic amino acid.
5) Method according to any one of items 1 to 4, wherein said To is included into said selected codon.
6) Method according to item 5, wherein said To is being removed from the target sequence upon insertion of the corrected codon at said locus by homologous recombination.
7) Method according to any one of items 1 to 6, wherein from 2 to 5 synonymous codons, preferably from 2 to 3, are introduced in the nucleic acid template to introduce mutations into the target polynucleotide sequence to prevent retargeting of the TALE-nuclease once the corrected codon is inserted by homologous recombination.
8) Method according to any one of items 1 to 7, wherein said TALE-nuclease is a heterodimer member that has to dimerize with a second TALE-monomer, such as a TALE-fok1 monomer.
9) Method according to item 8, wherein the selected codon is located in the spacer sequence located between the binding sequences of the first and second TALE monomers.
10) A allele specific TALE-nuclease A allele specific TALE-nuclease to target mutation causing E6V mutation in HBB comprising the following RVD sequence:
   NN-NN-NI-NN-NI-NI-NN-NG-HD-NG-NN-HD-HD-NN-NG-NG
11) A allele specific TALE-nuclease according to item 10, comprising the polypeptide sequence SEQ ID NO: 3.
12) A allele specific TALE-nuclease according to item 10 or 11 for use in therapy, especially gene therapy.
13) A allele specific TALE-nuclease according to any one of items 10 to 12 for the treatment of a genetic disorder.
14) A allele specific TALE-nuclease according to any one of items 10 to 13 for the treatment of a Hemoglobinopathy.
15) A allele specific TALE-nuclease according to any one of items 10 to 14 for the treatment of sickle cell anemia.
16) Method for mutating a cell line at different loci, wherein said method comprises the following steps:
   identifying a T (T₀) located at or at a distance less than 60 pb, preferably less than 30 pb of a selected codon to be corrected at said endogenous locus;
   identifying target sequence starting from said To in the 5′→3′ direction;
   providing nucleic acid templates homologous to said endogenous locus, encompassing said target sequences and comprising stop or modified codon(s), and optionally at synonymous codon(s) for insertion by homologous recombination at the different specific loci upon cleavage by said TALE-nucleases, wherein said corrected codon and said optional synonymous codon(s) introduce mutation(s) into said polynucleotide target sequence,
   providing nucleic acids encoding TALE-nucleases comprising RVD sequences which have been designed to bind the initial target sequences but which cannot bind said mutated target sequences when the stop or modified codons have been inserted by homologous recombination,
   introducing into the cell said nucleic acid templates comprising said stop or modified codons along with the nucleic acids encoding said TALE-nucleases;
   culturing the cells to allow expression of said TALE-nucleases and the insertion of said stop codons at the different loci;
   selecting the cells that have the stop codons inserted in their genomes at these loci.
17) Method for determining the “minimal peptidome” related to a cell function, or related to the survival a cell genome-wide in certain environmental conditions, said method comprising:
   inactivating a cell at different loci using the method according to item 16,
   culturing the cell over several generation to ensure maximal insertion of stop codons,
   isolating the surviving cells,
   determining which loci are mutated in the surviving cells and which are not;
   determining those loci that cannot be mutated alone or in combination by comparing the results obtained with different clones of surviving cells.
18) A kit for allele-specific codon modification at a locus in a cell, said kit comprising at least:
   a nucleic acid template comprising a TALE target sequence from an endogenous locus that has been mutated by the insertion of a modified codon, and
   a nucleic acid encoding a TALE-nuclease that has been designed such that the TALE nuclease that bind the original target sequence does not recognize said mutated target sequence when said modified codon has been inserted at said locus by homologous recombination.
19) A kit according to item 18 for use in therapy.
20)A kit according to item 18 or 19 for use in gene therapy.
21) A kit according to any one of items 18 to 20 for the treatment of a genetic disorder.
22) A kit according to any one of items 18 to 21 for the ex-vivo gene correction of blood cells.
23) A kit according to any one of items 18 to 22 for the treatment of a Hemoglobinopathy, such as b-thalassemia or sickle cell anemia.
24) A kit according to item 23, wherein said TALE-nuclease targets HBB.
25) A kit according to item 24, wherein said TALE-nuclease is according to any one of item 10 to 15.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only.

### Examples

### Example 1

### Design of Allele-specific TTR and HBB TALEN® and Corresponding DNA Template to Induce Cleavage of Pathological Allele Forms

TALE-nucleases enable the site-specific introduction of double-stranded breaks (DSBs) at precise loci in the genome with very high specificity. Repair of DSBs occurs largely through one of two pathways, non-homologous end joining (NHEJ) and homology directed repair (HDR). NHEJ is an error-prone pathway that often results in insertions or deletions (indels) whereas HDR uses a homologous DNA template to correctly repair the lesion by recombination. This homologous DNA template is normally provided by either the homologous chromosome or the sister chromatid, but it can also be exogenously-supplied as single-stranded oligonucleotides or as double-stranded DNA templates to introduce any genetic modifications encoded in the template DNA such as nucleotide changes to repair a defective gene or gene insertions. However, if the nuclease target site is present in the repair template, the nuclease will continue to cleave the locus and disrupt the genetic modifications encoded in the template DNA. Thus, exogenously-supplied repair templates would require removal of the target site, which is challenging when repairing coding and/or regulatory sequences. Here, another approach has been pursued by designing TALE-nucleases and corresponding DNA repair templates, in such a way that mutations can be introduced in the target site for said TALE-nuclease through the repair template in order to prevent retargeting functional alleles with minimal effects on gene expression.

Materials and Methods:

TALE-nuclease Reagents

TALEN® designates commercial grade Fok-1 based heterodimeric architecture of TALE-nuclease as described by Christian et al. [Targeting DNA double-strand breaks with TAL effector nucleases (2010) Genetics. 186(2):757-761] and manufactured by Cellectis SA (8 rue de la Croix Jarry, 75013 PARIS).

Here, TALEN® have been designed to target TTR and HBB mutated allele forms which preferentially target alleles that contain a thymidine (T) by taking advantage of the necessity of TALE binding a T at position 0 (T₀).

With respect to TTR, TALEN® was produced that could preferentially cleave the V3OM allele of transthyretin (TTR) characteristic of transthyretin amyloidosis by designing a left TTR-V30M-L1 and right TTR-V30M-R1 heterodimers (SEQ ID NO:1 and SEQ ID NO:2) harboring respectively the following RVD sequences:
HD-NG-NI-NN-NI-NG-NN-HD-NG-NN-NG-HD-HD-NG (TTR-V30M-L1), and
NN-NN-HD-HD-NI-HD-NI-NG-NG-NN-NI-NG-NN-NG (TTR-V30M-R1).

As shown in FIG. 2A, TTR-V30M-L1 was designed to selectively bind target sequence 5′—(T₀)GGCCACATTGATGG (SEQ ID NO:7), but not 5′ (G)GGCCACATTGATGG.

To assess the efficiency of these TALEN® heterodimers, stable isogenic cGPS HEK-293 cell lines were created using targeted integration that contained a copy of the WT (SEQ ID NO:9) or V30M (SEQ ID NO:10) allele polynucleotide sequence of TTR embedded between amplifiable sequences.

With respect to HBB, TALEN® that could preferentially cleave the E6V allele of beta-globin B (HBB) characteristic of sickle cell anemia was produced by designing the left and right heterodimers of SEQ ID NO:3 and SEQ ID NO:4, harboring respectively the following RVD sequences:
NN-NN-NI-NN-NI-NI-NN-NG-HD-NG-NN-HD-HD-NN-NG-NG (HBB-E6V-L1), and
HD-HD-NI-HD-NN-NG-NG-HD-NI-HD-HD-NG-NG-NN-HD-NG (HBB-E6V-R1).

As shown in FIG. 2B, HBB-E6V-L1 was designed to selectively bind target sequence 5′—(T₀) GGAGAAGTCTGCCGTT (SEQ ID NO:11), but not 5′ (A)GGCCACATTGATGG.

To assess the efficiency of these TALEN® heterodimers, SC-1 cells, a B cell line homozygous for the E6V sickle cell allele, or Raji cells, a B cell line homozygous for the WT allele of HBB were transfected with mRNA encoding these TALE-nucleases

Cells

Raji and SC-1 cells were purchased from ATCC (Manassas, Va., USA) and cultured in RPMI-1640 supplemented with 10% or 20% fetal bovine serum, respectively. The creation of stable cGPS HEK-293 isogenic cell lines using integration matrices that target the WT or V30M allele of TTR (SEQID 3 and SEQID 4) followed manufacturer's instructions (Cellectis Bioresearch). cGPS HEK-293 cells were cultured in DMEM supplemented with 10% fetal bovine serum.

Transfection

mRNAs encoding TALEN® were produced using the mMESSAGE mMACHINE T7 Kit (ThermoFisher Scientific) and purified using RNeasy Mini Spin Columns (Qiagen).

1×10⁶SC-1 or Raji cells were electroporated with 10 μg of TALEN mRNA per heterodimer using the Cytopulse Technology (PMID: 26015965). After 7 days of culture, genomic DNA was isolated using DNeasy Blood & Tissue Kit (Qiagen). 2.5×10⁵ cGPS HEK-293 cells harboring the WT or V30M allele of TTR were plated in a 12-well tissue culture plate. The next day, cells were transfected with 500 ng of TALEN mRNA per heterodimer using the TransIT-mRNA transfection kit (Mirus Bio). After 3 days of culture, genomic DNA was isolated using DNeasy Blood & Tissue Kit (Qiagen).

T7 Endonuclease Assay

PCR products surrounding the TALEN® cleavage site were amplified from genomic DNA and 50 ng of this product digested using T7 endonuclease 1 (T7E1). DNA fragments are separated on a 10% polyacrylamide gel and visualized by staining with SYBR green.

Results:

The digest of the PCR products on the polyacrylamide gels are reproduced on FIGS. 2A et 2B. FIG. 2A shows preferential cleavage of the TTR allele following transfection of mRNA encoding the TTR-V3OM TALEN® to cGPS HEK-293 cells harboring the WT or V30M allele of TTR. FIG. 2B shows preferential cleavage of the E6V allele of HBB in SC-1 cells is shown compared to the WT allele in Raji cells.

These results confirm that the both designed TALE-nucleases can discriminate wild type and mutated forms of the alleles and thus are allele-specific. This means that these TALE-nucleases can be used to repair the pathogenic alleles while lowering the probability of cleaving the functional alleles and re-cutting at the same locus upon gene repair. Such TALE-nuclease reagents thus represent safer reagents for gene therapy in view of treating Familial transthyretin amyloidosis and sickle cell anemia.

### Example 2

### Design of Specific TALE-nucleases and DNA Template to Induce HBB Repair at Codon 6 of Missense Mutation (A-to-T Transversion) by Synonymous Codon Substitution

### Materials and Methods:

Design of TALE-nuclease HBB-TALEN® and of its corresponding DNA template repair.

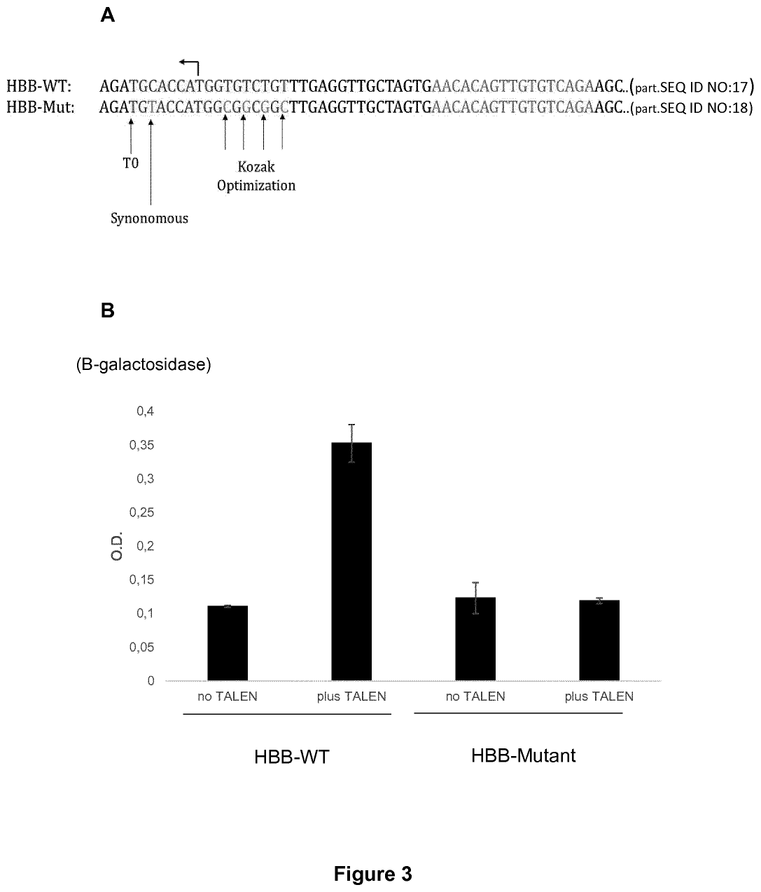

Specific HBB-T1 were designed by the inventors to target the beta-globin (HBB) locus (SEQ ID NO:17) in the 5′ UTR and a portion of the coding sequence of the gene (PMID: 25632877). Different target sequences were considered in this region and a series of mutations that inactivate TALE binding to the various target sites without altering expression of the functional gene were created. As the right arm of the TALE-nuclease recognizes a sequence in the 5′UTR through the coding sequence (SEQ ID NO:16), the inventors decided to introduce changes in the template sequence that would optimize the Kozak sequence within the 5′UTR, while introducing synonymous changes in the coding sequence. This strategy is shown in FIG. 3A. Based on these nucleotide changes, TALEN® were designed to discriminate between the pathological target sequence (HBB-WT—SEQ ID NO:17) and the repaired optimized sequence (HBB-mut—SEQ ID NO:18). Left and right resulting TALEN HBB-T1 heterodimers are characterized by the sequences mentioned in Table 1:

TABLE 1
Sequences related to HBB-TALEN of Example 2 and 3

| Name | Type | Sequence |
|---|---|---|
| HBB-T1-L1 | Polypeptide | MGDPKKKRKVIDYPYDVPDYAIDIADPIRSRTPSP |
| | SEQ ID NO: 5 | ARELLPGPQPDGVQPTADRGVSPPAGGPLDGLPAR |
| | | MGDPKKKRKVIDYPYDVPDYAIDIADPIRSRTPSP |
| | | ARELLPGPQPDGVQPTADRGVSPPAGGPLDGLPAR |
| | | RTMSRTRLPSPPAPSPAFSAGSFSDLLRQFDPSLF |
| | | NTSLFDSLPPFGAHHTEAATGEWDEVQSGLRAADA |
| | | PPPTMRVAVTAARPPRAKPAPRRRAAQPSDASPAA |
| | | QVDLRTLGYSQQQQEKIKPKVRSTVAQHHEALVGH |
| | | GFTHAHIVALSQHPAALGTVAVKYQDMIAALPEAT |
| | | HEAIVGVGKQWSGARALEALLTVAGELRGPPLQLD |
| | | TGQLLKIAKRGGVTAVEAVHAWRNALTGAPLNLTP |
| | | EQVVAIASHDGGKQALETVQRLLPVLCQAHGLTPQ |
| | | QVVAIASNGGGKQALETVQRLLPVLCQAHGLTPQQ |
| | | VVAIASNNGGKQALETVQRLLPVLCQAHGLTPEQV |
| | | VAIASNIGGKQALETVQALLPVLCQAHGLTPEQVV |
| | | AIASHDGGKQALETVQRLLPVLCQAHGLTPEQVVA |
| | | IASNIGGKQALETVQALLPVLCQAHGLTPEQVVAI |
| | | ASNMGGKQALETVQRLLPVLCQAHGLTPEQVVAIA |
| | | SNIGGKQALETVQALLPVLCQAHGLTPEQVVAIAS |
| | | NIGGKQALETVQALLPVLCQAHGLTPEQVVAIASH |
| | | DGGKQALETVQRLLPVLCQAHGLTPQQVVAIASLP |
| | | GGKQALETVQRLLPVLCQAHGLTPQQVVAIASNNG |
| | | GKQALETVQRLLPVLCQAHGLTPQQVVAIASNGGG |
| | | KQALETVQRLLPVLCQAHGLTPQQVVAIASNNGGK |
| | | QALETVQRLLPVLCQAHGLTPQQVVAIASNGGGKQ |
| | | ALETVQRLLPVLCQAHGLTPQQVVAIASNGGGRPA |
| | | LESIVAQLSRPDPALAALTNDHLVALACLGGRPAL |
| | | DAVKKGLGDPISRSQLVKSELEEKKSELRHKLKYV |
| | | PHEYIELIEIARNSTQDRILEMKVMEFFMKVYGYR |
| | | GKHLGGSRKPDGAIYTVGSPIDYGVIVDTKAYSGG |
| | | YNLPIGQADEMQRYVEENQTRNKHINPNEWWKVYP |
| | | SSVTEFKFLFVSGHFKGNYKAQLTRLNHITNCNGA |
| | | VLSVEELLIGGEMIKAGTLTLEEVRRKFNNGEINF |
| | | AAD |
| | RVD | HD-NG-NN-NI-HD-NI-NM-NI-NI-HD-LP- |
| | | NN-NG-NN-NG-NG |
| | Target | CTGACACAACTGTGTT |
| | sequence | |
| | SEQ ID NO. 15 | |
| HBB-T1-R1 | Polypeptide | MGDPKKKRKVIDKETAAAKFERQHMDSIDIADPIR |
| | SEQ ID NO: 6 | SRTPSPARELLPGPQPDGVQPTADRGVSPPAGGPL |
| | | MGDPKKKRKVIDKETAAAKFERQHMDSIDIADPIR |
| | | SRTPSPARELLPGPQPDGVQPTADRGVSPPAGGPL |
| | | DGLPARRTMSRTRLPSPPAPSPAFSAGSFSDLLRQ |
| | | FDPSLFNTSLFDSLPPFGAHHTEAATGEWDEVQSG |
| | | LRAADAPPPTMRVAVTAARPPRAKPAPRRRAAQPS |
| | | DASPAAQVDLRTLGYSQQQQEKIKPKVRSTVAQHH |
| | | EALVGHGFTHAHIVALSQHPAALGTVAVKYQDMIA |
| | | ALPEATHEAIVGVGKQWSGARALEALLTVAGELRG |
| | | PPLQLDTGQLLKIAKRGGVTAVEAVHAWRNALTGA |
| | | PLNLTPQQVVAIASNNGGKQALETVQRLLPVLCQA |
| | | HGLTPEQVVAIASHDGGKQALETVQRLLPVLCQAH |
| | | GLTPEQVVAIASNIGGKQALETVQALLPVLCQAHG |
| | | LTPEQVVAIASHDGGKQALETVQRLLPVLCQAHGL |
| | | TPEQVVAIASHDGGKQALETVQRLLPVLCQAHGLT |
| | | PEQVVAIASNIGGKQALETVQALLPVLCQAHGLTP |
| | | QQVVAIASNGGGKQALETVQRLLPVLCQAHGLTPQ |
| | | QVVAIASNNGGKQALETVQRLLPVLCQAHGLTPQQ |
| | | VVAIASNNGGKQALETVQRLLPVLCQAHGLTPQQV |
| | | VAIASNGGGKQALETVQRLLPVLCQAHGLTPQQVV |
| | | AIASNNGGKQALETVQRLLPVLCQAHGLTPQQVVA |
| | | IASNGGGKQALETVQRLLPVLCQAHGLTPEQVVAI |
| | | ASHDGGKQALETVQRLLPVLCQAHGLTPQQVVAIA |
| | | SNGGGKQALETVQRLLPVLCQAHGLTPQQVVAIAS |
| | | NNGGKQALETVQRLLPVLCQAHGLTPQQVVAIASN |
| | | GGGRPALESIVAQLSRPDPALAALTNDHLVALACL |
| | | GGRPALDAVKKGLGDPISRSQLVKSELEEKKSELR |
| | | HKLKYVPHEYIELIEIARNSTQDRILEMKVMEFFM |
| | | KVYGYRGKHLGGSRKPDGAIYTVGSPIDYGVIVDT |
| | | KAYSGGYNLP1GQADEMQRYVEENQTRNKHINPNE |
| | | WWKVYPSSVTEFKFLFVSGHFKGNYKAQLTRLNHI |
| | | TNCNGAVLSVEELLIGGEM1KAGTLTLEEVRRKFN |
| | | NGEINFAAD |
| | RVD | NN-HD-NI-HD-HD-NI-NG-NN-NN-NG-NN- |
| | | NG-HD-NG-NN-NG |
| | Target | GCACCATGGTGTCTGT |
| | sequence | |
| | SEQ ID NO. 16 | |
| HBB-T2-L | Polypeptide | MGDPKKKRKVIDYPYDVPDYAIDIADLRTLGYSQQ |
| | SEQ ID NO: 93 | QQEKIKPKVRSTVAQHHEALVGHGFTHAHIVALSQ |
| | | MGDPKKKRKVIDYPYDVPDYAIDIADLRTLGYSQQ |
| | | QQEKIKPKVRSTVAQHHEALVGHGFTHAHIVALSQ |
| | | HPAALGTVAVKYQDMIAALPEATHEAIVGVGKQWS |
| | | GARALEALLTVAGELRGPPLQLDTGQLLKIAKRGG |
| | | VTAVEAVHAWRNALTGAPLNLTPQQVVAIASNNGG |
| | | KQALETVQRLLPVLCQAHGLTPEQVVAIASHDGGK |
| | | QALETVQRLLPVLCQAHGLTPQQVVAIASNGGGKQ |
| | | ALETVQRLLPVLCQAHGLTPQQVVAIASNGGGKQA |
| | | LETVQRLLPVLCQAHGLTPEQVVAIASNIGGKQAL |
| | | ETVQALLPVLCQAHGLTPEQVVAIASHDGGKQALE |
| | | TVQRLLPVLCQAHGLTPEQVVAIASNIGGKQALET |
| | | VQALLPVLCQAHGLTPQQVVAIASNGGGKQALETV |
| | | QRLLPVLCQAHGLTPQQVVAIASNGGGKQALETVQ |
| | | RLLPVLCQAHGLTPQQVVAIASNGGGKQALETVQR |
| | | LLPVLCQAHGLTPQQVVAIASNNGGKQALETVQRL |
| | | LPVLCQAHGLTPEQVVAIASHDGGKQALETVQRLL |
| | | PVLCQAHGLTPQQVVAIASNGGGKQALETVQRLLP |
| | | VLCQAHGLTPQQVVAIASNGGGKQALETVQRLLPV |
| | | LCQAHGLTPEQVVAIASHDGGKQALETVQRLLPVL |
| | | CQAHGLTPQQVVAIASNGGGRPALESIVAQLSRPD |
| | | PALAALTNDHLVALACLGGRPALDAVKKGLGDPIS |
| | | RSQLVKSELEEKKSELRHKLKYVPHEYIELIEIAR |
| | | NSTQDRILEMKVMEFFMKVYGYRGKHLGGSRKPDG |
| | | AIYTVGSPIDYGVIVDTKAYSGGYNLPIGQADEMQ |
| | | RYVEENQTRNKH1NPNEWWKVYPSSVTEFKFLFVS |
| | | GHFKGNYKAQLTRLNHITNCNGAVLSVEELLIGGE |
| | | MIKAGTLTLEEVRRKFNNGEINFAAD |
| | RVD | NN-HD-NG-NG-NI-HD-NI-NG-NG-NG-NN- |
| | | HD-NG-NG-HD |
| | Target | TGCTTACATTTGCTTCT |
| | sequence | |
| | SEQ ID NO: 86 | |
| HBB-T2-R | Polypeptide | MGDPKKKRKVIDYPYDVPDYAIDIADLRTLGYSQQ |
| | SEQ ID NO: 94 | QQEKIKPKVRSTVAQHHEALVGHGFTHAHIVALSQ |
| | | MGDPKKKRKVIDYPYDVPDYAIDIADLRTLGYSQQ |
| | | QQEKIKPKVRSTVAQHHEALVGHGFTHAHIVALSQ |
| | | HPAALGTVAVKYQDMIAALPEATHEAIVGVGKQWS |
| | | GARALEALLTVAGELRGPPLQLDTGQLLKIAKRGG |
| | | VTAVEAVHAWRNALTGAPLNLTPQQVVAIASNNGG |
| | | KQALETVQRLLPVLCQAHGLTPQQVVAIASNGGGK |
| | | QALETVQRLLPVLCQAHGLTPQQVVAIASNGGGKQ |
| | | ALETVQRLLPVLCQAHGLTPQQVVAIASNGGGKQA |
| | | LETVQRLLPVLCQAHGLTPQQVVAIASNNGGKQAL |
| | | ETVQRLLPVLCQAHGLTPEQVVAIASNIGGKQALE |
| | | TVQALLPVLCQAHGLTPQQVVAIASNNGGKQALET |
| | | VQRLLPVLCQAHGLTPQQVVAIASNNGGKQALETV |
| | | QRLLPVLCQAHGLTPQQVVAIASNGGGKQALETVQ |
| | | RLLPVLCQAHGLTPQQVVAIASNGGGKQALETVQR |
| | | LLPVLCQAHGLTPQQVVAIASNNGGKQALETVQRL |
| | | LPVLCQAHGLTPEQVVAIASHDGGKQALETVQRLL |
| | | PVLCQAHGLTPQQVVAIASNGGGKQALETVQRLLP |
| | | VLCQAHGLTPEQVVAIASNIGGKQALETVQALLPV |
| | | LCQAHGLTPQQVVAIASNNGGKQALETVQRLLPVL |
| | | CQAHGLTPQQVVAIASNGGGRPALESIVAQLSRPD |
| | | PALAALTNDHLVALACLGGRPALDAVKKGLGDPIS |
| | | RSQLVKSELEEKKSELRHKLKYVPHEYIELIEIAR |
| | | NSTQDRILEMKVMEFFMKVYGYRGKHLGGSRKPDG |
| | | AIYTVGSPIDYGVIVDTKAYSGGYNLPIGQADEMQ |
| | | RYVEENQTRNKHINPNEWWKVYPSSVTEFKFLFVS |
| | | GHFKGNYKAQLTRLNHITNCNGAVLSVEELLIGGE |
| | | MIKAGTLTLEEVRRKFNNGEINFAAD |
| | RVD | NN-NG-NG-NG-NN-NI-NN-NN-NG-NG-NN- |
| | | HD-NG-NI-NN |
| | Target | TGTTTGAGGTTGCTAGT |
| | sequence | |
| | SEQ ID NO: 88 | |
| HBB-T3-L | Polypeptide | MGDPKKKRKVIDYPYDVPDYAIDIADLRTLGYSQQ |
| | SEQ ID NO: 95 | QQEKIKPKVRSTVAQHHEALVGHGFTHAHIVALSQ |
| | | MGDPKKKRKVIDYPYDVPDYAIDIADLRTLGYSQQ |
| | | QQEKIKPKVRSTVAQHHEALVGHGFTHAHIVALSQ |
| | | HPAALGTVAVKYQDMIAALPEATHEAIVGVGKQWS |
| | | GARALEALLTVAGELRGPPLQLDTGQLLKIAKRGG |
| | | VTAVEAVHAWRNALTGAPLNLTPQQVVAIASNGGG |
| | | KQALETVQRLLPVLCQAHGLTPEQVVAIASNIGGK |
| | | QALETVQALLPVLCQAHGLTPEQVVAIASHDGGKQ |
| | | ALETVQRLLPVLCQAHGLTPEQVVAIASNIGGKQA |
| | | LETVQALLPVLCQAHGLTPQQVVAIASNGGGKQAL |
| | | ETVQRLLPVLCQAHGLTPQQVVAIASNGGGKQALE |
| | | TVQRLLPVLCQAHGLTPQQVVAIASNGGGKQALET |
| | | VQRLLPVLCQAHGLTPQQVVAIASNNGGKQALETV |
| | | QRLLPVLCQAHGLTPEQVVAIASHDGGKQALETVQ |
| | | RLLPVLCQAHGLTPQQVVAIASNGGGKQALETVQR |
| | | LLPVLCQAHGLTPQQVVAIASNGGGKQALETVQRL |
| | | LPVLCQAHGLTPEQVVAIASHDGGKQALETVQRLL |
| | | PVLCQAHGLTPQQVVAIASNGGGKQALETVQRLLP |
| | | VLCQAHGLTPQQVVAIASNNGGKQALETVQRLLPV |
| | | LCQAHGLTPEQVVAIASNIGGKQALETVQALLPVL |
| | | CQAHGLTPQQVVAIASNGGGRPALESIVAQLSRPD |
| | | PSGSGSGGDPISRSQLVKSELEEKKSELRHKLKYV |
| | | PHEYIELIEIARNSTQDRILEMKVMEFFMKVYGYR |
| | | GKHLGGSRKPDGAIYTVGSPIDYGVIVDTKAYSGG |
| | | YNLPIGQADEMQRYVEENQTRNKHINPNEWWKVYP |
| | | SSVTEFKFLFVSGHFKGNYKAQLTRLNHITNCNGA |
| | | VLSVEELLIGGEMIKAGTLTLEEVRRKFNNGEINF |
| | | AAD |
| | RVD | NG-NI-HD-NI-NG-NG-NG-NN-HD-NG-NG- |
| | | HD-NG-NN-NI |
| | Target | TTACATTTGCTTCTGAC |
| | sequence | |
| | SEQ ID NO: 90 | |
| HBB-T3-L | Polypeptide | MGDPKKKRKVIDYPYDVPDYAIDIADLRTLGYSQQ |
| | SEQ ID NO: 96 | QQEKIKPKVRSTVAQHHEALVGHGFTHAHIVALSQ |
| | | MGDPKKKRKVIDYPYDVPDYAIDIADLRTLGYSQQ |
| | | QQEKIKPKVRSTVAQHHEALVGHGFTHAHIVALSQ |
| | | HPAALGTVAVKYQDMIAALPEATHEAIVGVGKQWS |
| | | GARALEALLTVAGELRGPPLQLDTGQLLKIAKRGG |
| | | VTAVEAVHAWRNALTGAPLNLTPQQVVAIASNNGG |
| | | KQALETVQRLLPVLCQAHGLTPQQVVAIASNGGGK |
| | | QALETVQRLLPVLCQAHGLTPQQVVAIASNGGGKQ |
| | | ALETVQRLLPVLCQAHGLTPQQVVAIASNGGGKQA |
| | | LETVQRLLPVLCQAHGLTPQQVVAIASNNGGKQAL |
| | | ETVQRLLPVLCQAHGLTPEQVVAIASNIGGKQALE |
| | | TVQALLPVLCQAHGLTPQQVVAIASNNGGKQALET |
| | | VQRLLPVLCQAHGLTPQQVVAIASNNGGKQALETV |
| | | QRLLPVLCQAHGLTPQQVVAIASNGGGKQALETVQ |
| | | RLLPVLCQAHGLTPQQVVAIASNGGGKQALETVQR |
| | | LLPVLCQAHGLTPQQVVAIASNNGGKQALETVQRL |
| | | LPVLCQAHGLTPEQVVAIASHDGGKQALETVQRLL |
| | | PVLCQAHGLTPQQVVAIASNGGGKQALETVQRLLP |
| | | VLCQAHGLTPEQVVAIASNIGGKQALETVQALLPV |
| | | LCQAHGLTPQQVVAIASNNGGKQALETVQRLLPVL |
| | | CQAHGLTPQQVVAIASNGGGRPALESIVAQLSRPD |
| | | PSGSGSGGDPISRSQLVKSELEEKKSELRHKLKYV |
| | | PHEYIELIEIARNSTQDRILEMKVMEFFMKVYGYR |
| | | GKHLGGSRKPDGAIYTVGSPIDYGVIVDTKAYSGG |
| | | YNLPIGQADEMQRYVEENQTRNKHINPNEWWKVYP |
| | | SSVTEFKFLFVSGHFKGNYKAQLTRLNHITNCNGA |
| | | VLSVEELLIGGEMIKAGTLTLEEVRRKFNNGEINF |
| | | AAD |
| | RVD | NN-NG-NG-NG-NN-NI-NN-NN-NG-NG-NN- |
| | | HD-NG-NI-NN |
| | Target | TGTTTGAGGTTGCTAGT |
| | sequence | |
| | SEQ ID NO: 92 | |
| HBB-in-out | SEQ ID NO: 5 | ACGTTSACCTTKCCCCACA |
| PCR-R6 | | |
| degenerated | | |
| primer | | |
| HBB-in-out | SEQ ID NO: 6 | TCGTTACCAAGCTGTGATTCC |
| PCR-F6 | | |

TALE-nuclease cleavage was assayed using the extrachromosomal single-stranded annealing (SSA) assay.

Extrachromosomal SSA Assay

Around 2E4 293FT cells were transfected with 100 ng of each arm of TALEN expression vectors and the reporter plasmid containing the wild-type or mutant sequence using Lipofectamine (ThermoFisher). β-galactosidase activity in the cells was assessed 72 hours post-transfection using mammalian beta-galactosidase assay kit (ThermoFisher

Scientific) and optical density measured at 420 nm (BMG Labtech). The plasmid construct that contains the lacZ reporter gene interrupted by the TALE target site was transfected to 293FT cells along with TALEN expression vectors. Cleavage by the TALEN will stimulate repair by SSA to create an intact lacZ gene. Using this assay, cells that received a plasmid vector that contained the wild-type version of the HBB TALE binding site were shown to produce β-galactosidase in the presence of TALEN, whereas those that received the mutant version of the HBB TALE binding site did not produce β-galactosidase in the presence of TALEN (FIG. 3B).

Co-transfection with AAV6 Vectors

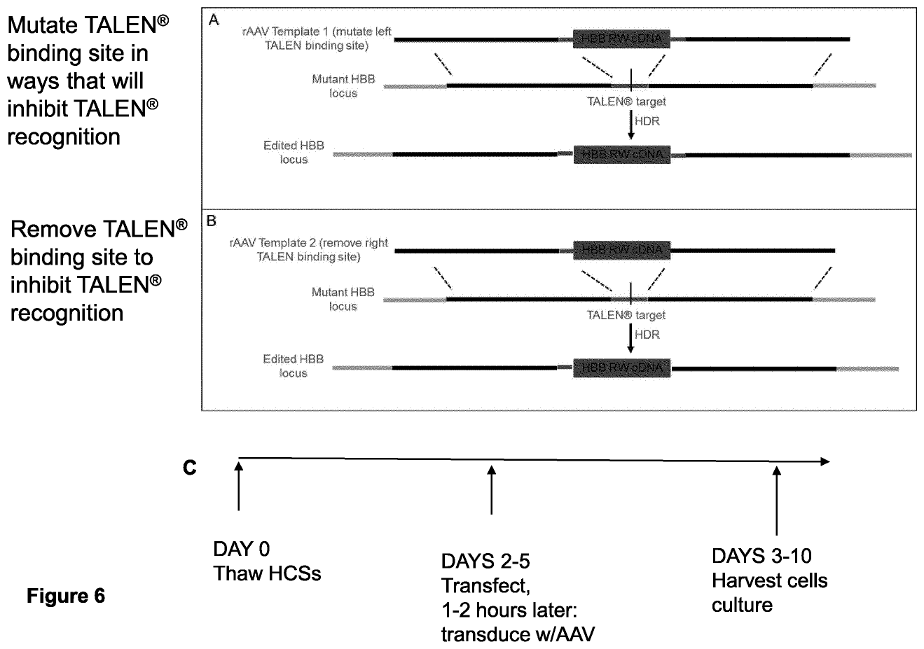

AAV6 vectors were designed and prepared to integrate the previous mutated target sequence by HDR (SEQ ID NO:19). AAV stocks were produced by triple transfection of AAV vector, serotype helper, and adenoviral helper plasmids in HEK 293T cells. Transfected cells were collected 48 hours later, lysed by freeze-thaw, benzonase-treated, and purified over iodixanol density gradient as previously described (Khan, I F et al. (2011) AAV-mediated gene targeting methods for human cells. Nat Protoc. 6:482-501). Shortly after TALE-nucleases mRNA transfection , cells were transduced with AAV as outline in FIG. 6C.

HSCs

Mobilized peripheral blood stem/progenitor cells (AllCells, LLC) were thawed and cultured in StemSpan serum-free expansion medium (SFEM) II (StemCell Technologies Inc) supplemented with CD34+ expansion supplement (StemCell Technologies Inc), BIT9500 serum substitute (StemCell Technologies Inc), Sodium Pyruvate (Gibco) and penicillin/streptomycin (Gibco). 5 days later, 2×106 cells were electroporated with 10 μg of TALEN mRNA per arm using the Cytopulse Technology (PMID: 26015965). mRNAs were produced using the mMESSAGE mMACHINE T7 Kit (ThermoFisher Scientific) and purified using RNeasy Mini Spin Columns (Qiagen). Recombinant AAV6 containing the HBB repair template, produced by Vigene (Rockville, Md.), was added to transfected cells at 1×10⁵ viral genomes/cell. After 8 days of culture, genomic DNA was isolated using

DNeasy Blood & Tissue Kit (Qiagen).

PCR

Modified alleles were determined using in-out PCR in which one primer anneals within the re-written HBB cDNA sequence in the repair matrix and another anneals outside of the homology arms and compared to a PCR using primers that anneal outside of the homology arms near the HBB locus. A 35-cycle PCR reaction was performed using 50 ng of genomic DNA for both PCRs.

A qPCR assay was also used to quantify modified alleles using primers that preferentially recognize the modified allele from the wild-type allele and normalized to the ACTB locus. 50 ng of genomic DNA was used in a PowerUp SYBR green (ThermoFisher Scientific) qPCR reaction and detected in CFX96 Touch Real-Time PCR Detection System (Bio-Rad). The percent of modified alleles was determined using the delta delta Ct method.

Results:

As shown in the extrachromosomal assay of FIG. 3B, HBB-mut TALEN® was found to efficiently discriminate HBB WT target sequence and the mutated target sequence in which a synonymous codon has been introduced. These results suggest that the engineered mutations in the HBB TALE binding site inhibits cleavage by the HBB TALEN.

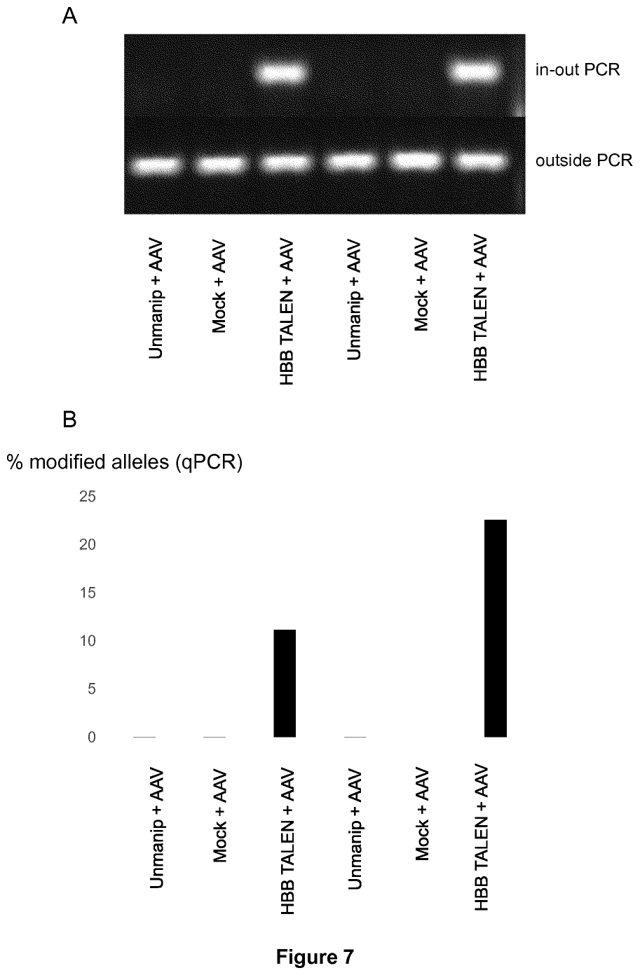

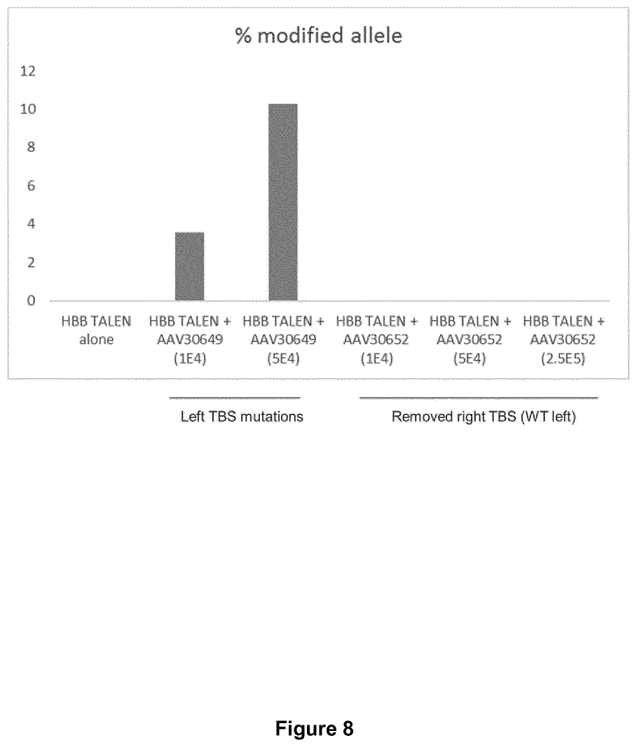

Next the engineered mutations in the HBB TALE binding site have been assayed to check whether the would permit repair of the HBB locus when delivered as a donor repair template using recombinant AAV (rAAV) along with HBB-mut TALEN® delivered as mRNA. The repair template delivered by rAAV contains a re-written version of the HBB cDNA surrounded by 300 bp homologies that centered around the DSB along with the engineered mutations in the TALE binding site. HSCs were transfected with 5 μg per arm of TALEN mRNA per 1×10⁶ cells followed by transduction with rAAV6 delivering the HBB repair template. In-out PCR was used to confirm modification of the HBB allele and compared to amplification of a genomic region outside of the HBB locus which revealed extensive modification of the HBB locus in HSCs treated with HBB TALEN plus rAAV6 (FIG. 7A). In addition, a qPCR assay using primers that selectively amplified the modified locus versus the wild-type locus confirmed the presence of modified alleles in HSCs treated with TALEN plus rAAV6 (FIG. 7B).

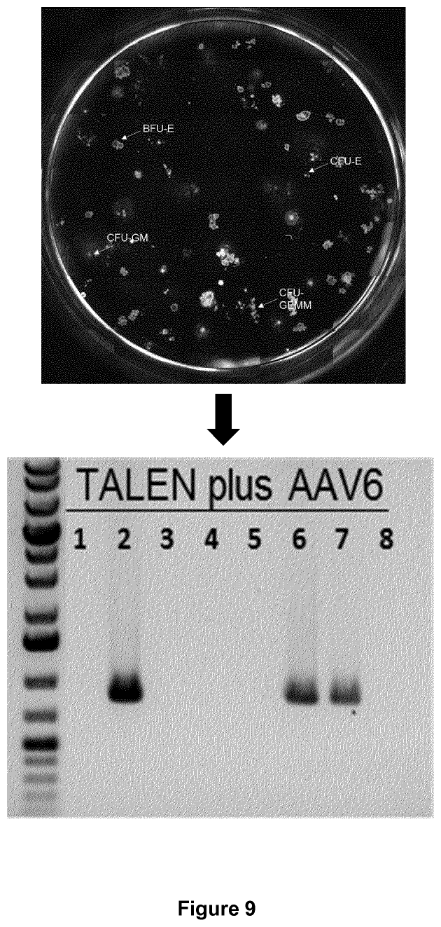

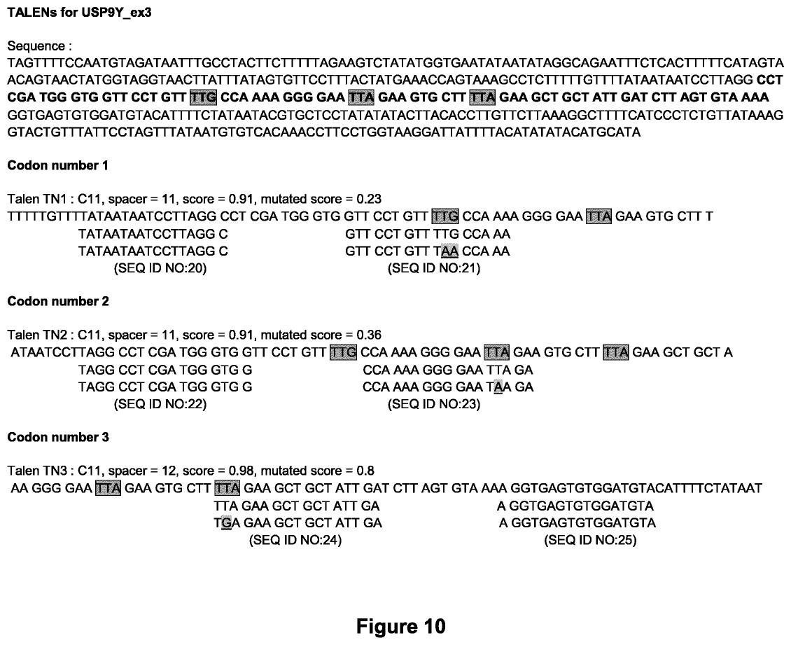

In order to insure the clinical use of edited HSPC, their differentiation potential was evaluated using a Colony Forming Unit (CFU) assay on methylcellulose (according to manufacturer, STEMCELL Technologies). HSPC cells were seeded either right after thawing (Thw) or 2 days after nucleofection with either no mRNA (P), 2 μg of GFP mRNA (GFP), 3+3 μg of TALEN® mRNAs unit, with or without nucleofection (UT). The methylcellulose differentiation assay showed no significant difference between samples demonstrating that edited HSPC can differentiate efficiently in every colony type. CFU assay was also used to assess allelic disruption, colonies were picked, their genomic DNA extracted and nucleases target sites were PCR amplified and sequenced (FIG. 9.

### Example 3

### Design of Further Specific TALE-nucleases and DNA Template to Induce HBB Repair and Corresponding DNA Template Repair Involving Mutation in the T0 of the Specific TALE Target Sequence

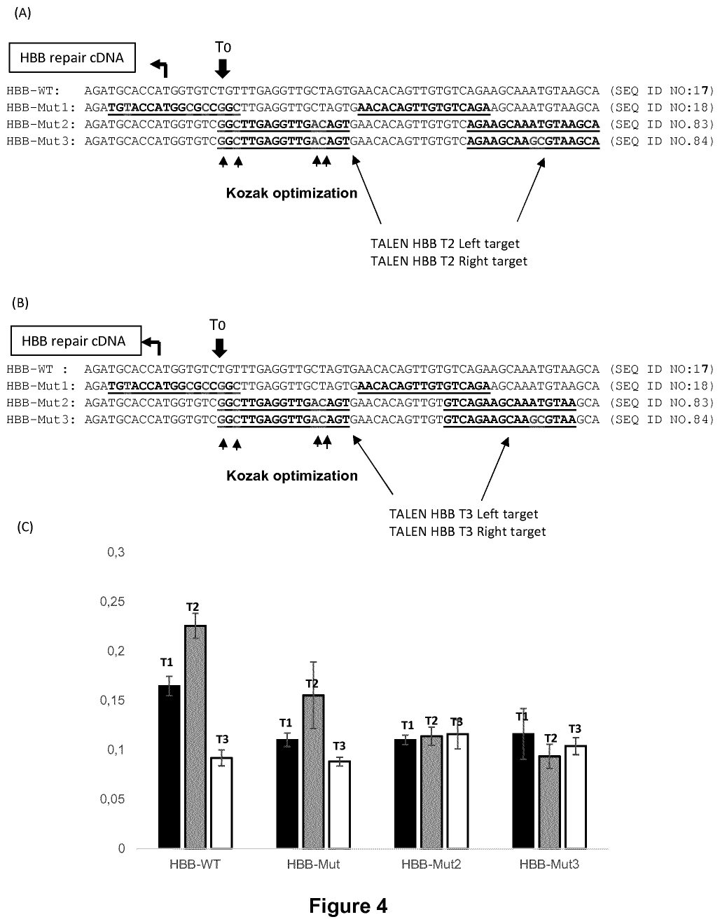

Specific HBB TALEN were designed by the inventors to target the beta-globin (HBB) locus in the 5′ UTR. Different target sequences were considered in this region and a series of mutations have been introduced in the polynucleotide template to be used for the site directed insertion of a functional HBB cDNA. The goal of these mutations in this template, upstream the coding sequence, was two fold : (1) to remove the T0 nucleotide from the initial TALE target sequence in order to prevent recutting once the cDNA is inserted at the locus, and (2) introduce further mutations to increase mismatch with the TALE while optimizing Kozak sequences. These mutations had to be introduced without altering expression of the introduced functional copy of the HBB gene. As both arms of the TALE-nucleases recognize a sequence in the 5′UTR, the inventors decided to introduce changes in the template sequence that would optimize the Kozak sequence within the 5′UTR. This strategy is shown in FIG. 4. Based on these nucleotide changes, TALEN® were designed to discriminate between the wild type target sequence (HBBWT—SEQ ID NO: 13) and the repaired optimized sequence HBB-mut2 (SEQ ID NO: 83) or HBB-mut3 (SEQ ID NO: 84). The sequences related to the first and second TALEN pairs (HBB T2 L/R and HBB T3 L/R are reported in Table 1.

Characterization of the cleavage obtainable with the above TALE-nucleases into the wild-type or the repaired optimized (mutated) sequences was performed as described in example 2. Using this assay, cells that received a plasmid vector that contained the wild-type version of the HBB TALE binding site were shown to produce β-galactosidase in the presence of TALEN, whereas those that received the mutant version of the HBB TALE binding site did not produce β-galactosidase in the presence of TALEN (FIG. 4C). Next both TALEN pairs have been assayed to check whether they would permit repair of the HBB locus when delivered together as mRNA with a donor repair template (from Example 2) using recombinant AAV (rAAV). The repair template delivered by rAAV contained a re-written version of the HBB cDNA surrounded by 300 bp homologies that centered around the DSB along with the engineered HBB-Mut mutations. HSCs were transfected and handled as described in Example 2.

Modified alleles were determined using in-out PCR in which one degenerated primer anneals within both, the re-written HBB cDNA brought the repair matrix, or the endogenous sequence (HBB-in-out PCR-R6, SEQ ID NO:5) and another annealed outside of the homology arms (HBB-in-out PCR-F6, SEQ ID NO:6).

PCR amplification of the HBB locus was performed on genomic DNA using Phusion High-Fidelity PCR Master Mix with HF Buffer (NEB, #M0531S) according to the manufacturer instructions. PCR products were subclone using the CloneJET PCR Cloning Kit (Thermo Scientific, #K1231) according to the manufacturer instructions. Plasmid DNA was extracted from individual colonies and analyzed via Sanger sequencing. Sequences were then classified as wild-type, Indels (containing small insertions/deletions at the TALEN cleavage site) or HR (containing the re-written HBB cDNA).

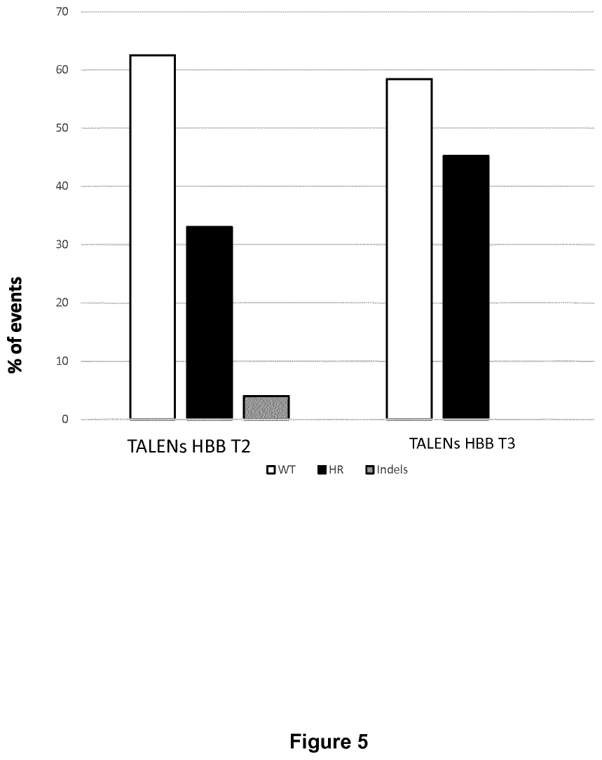

The results of this analysis were plotted in the diagram of FIG. 5.

These results confirmed that presence of modified alleles in HSCs treated with the TALE-nucleases HBB T2 L/R and HBB T3 L/R, plus rAAV6 comprising either HBB-mut1 (SEQ ID NO: 18). These data demonstrate that a mutated template could be integrated at high efficiency with almost no indels.

### Example 4: Allele-specific codon substitution by stop codon using HDR for multiplexing gene inactivation

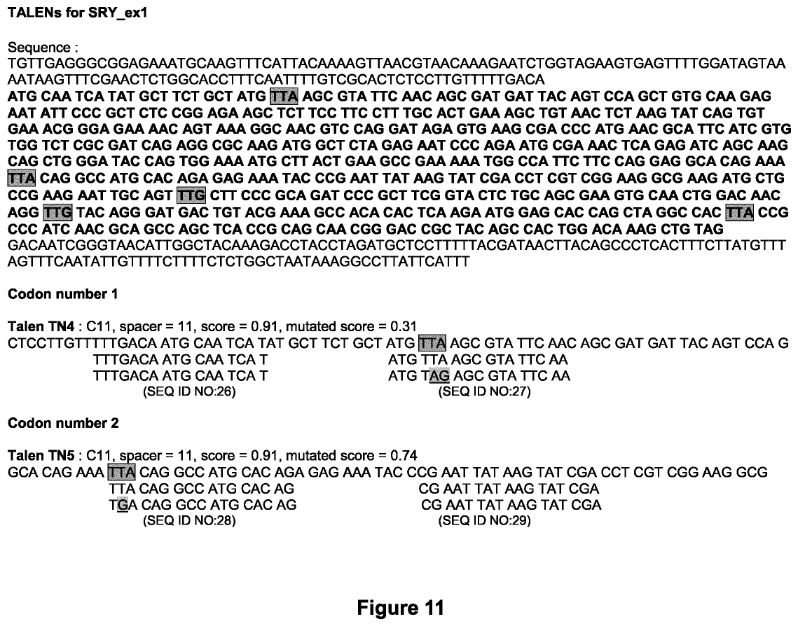

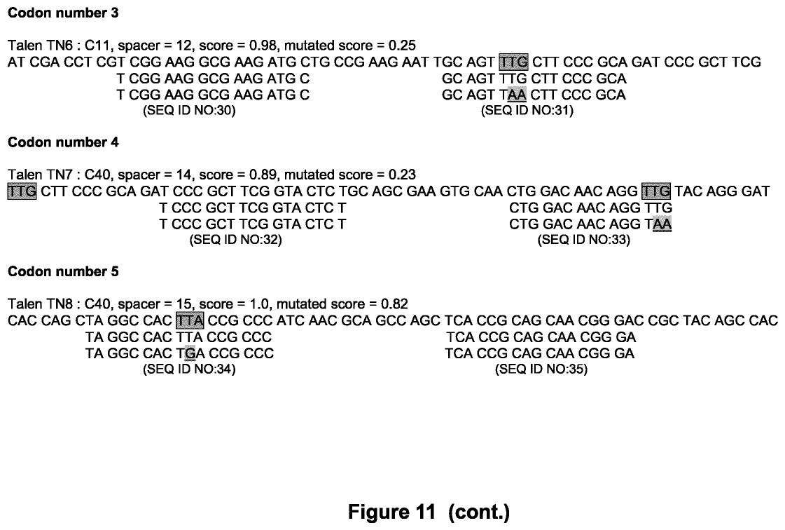

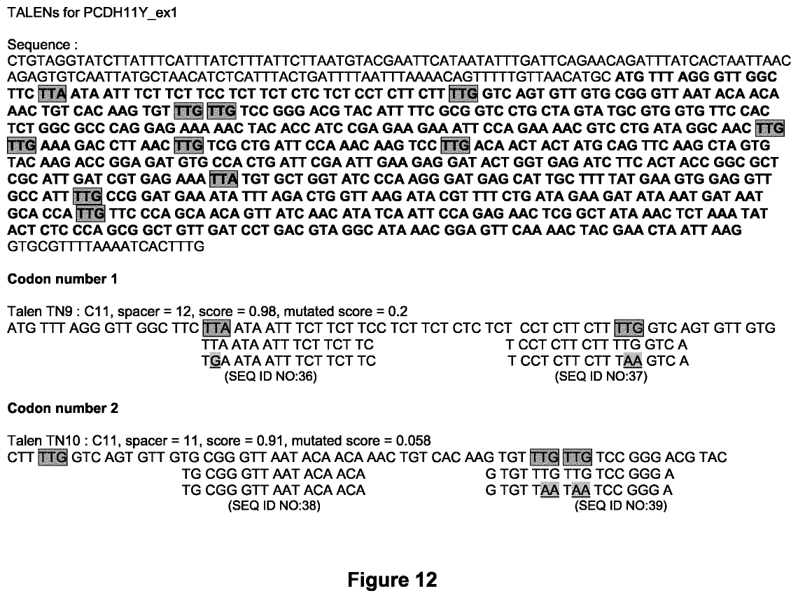

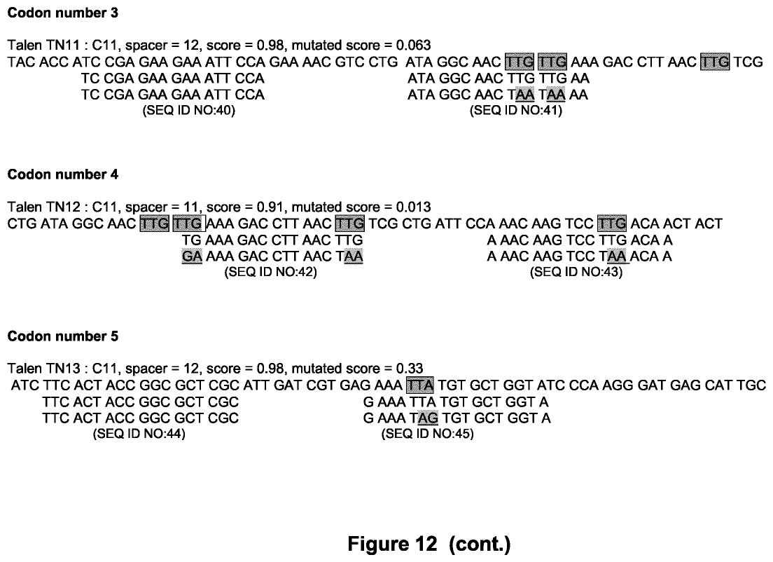

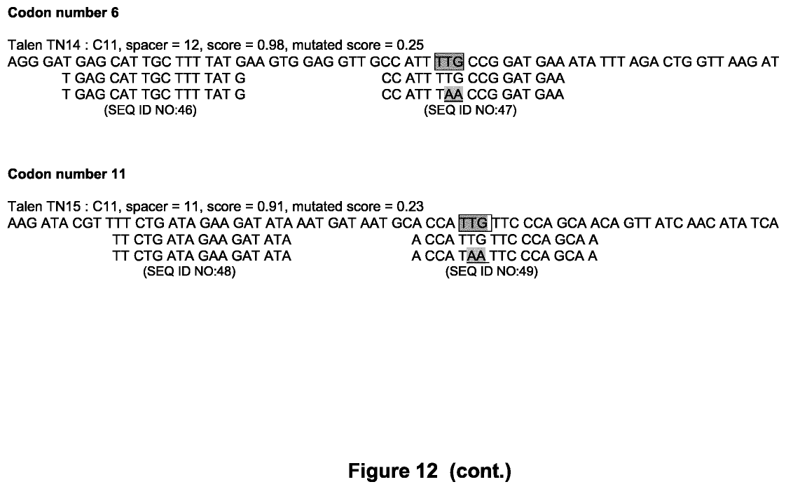

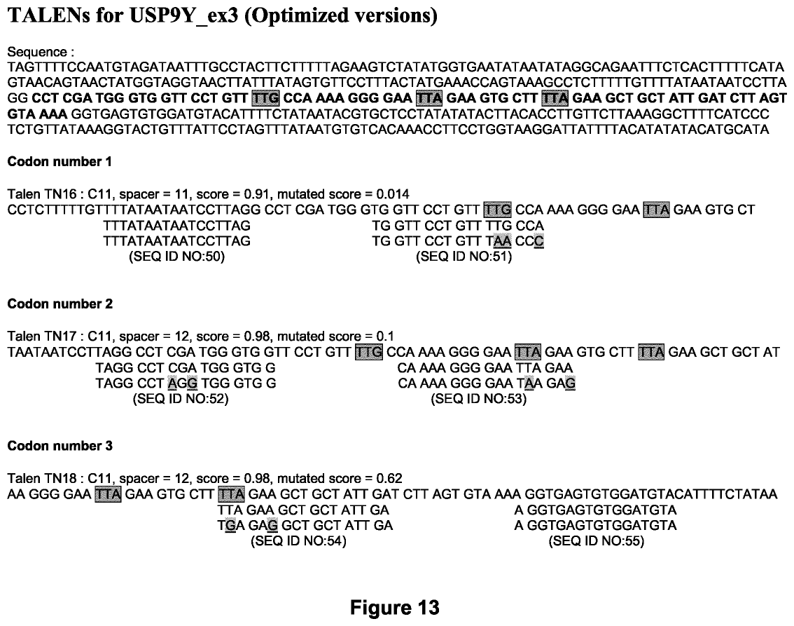

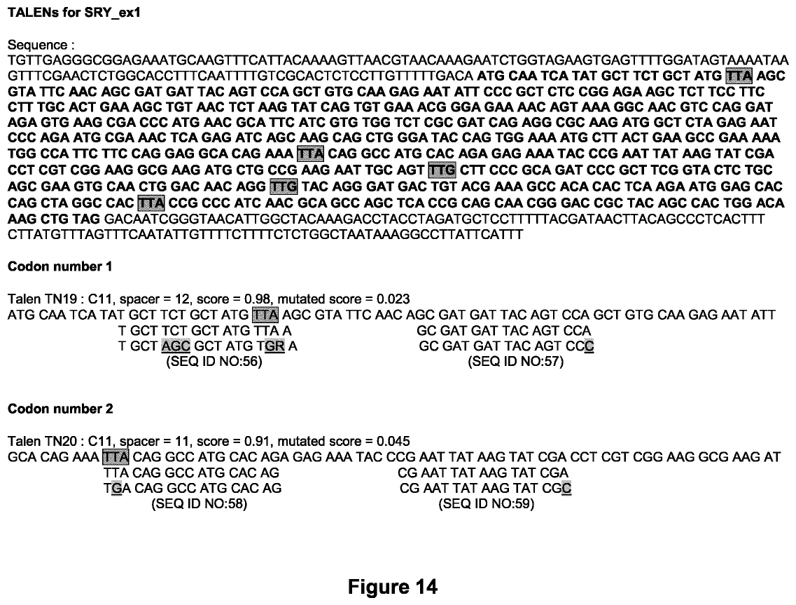

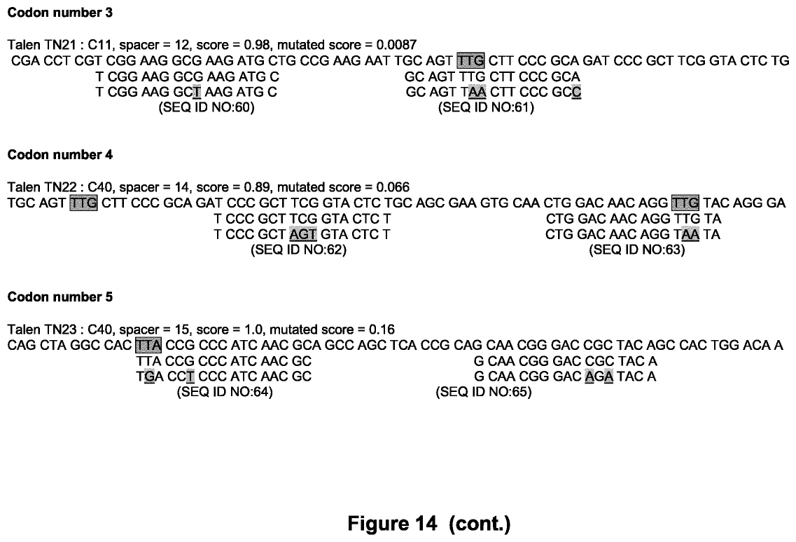

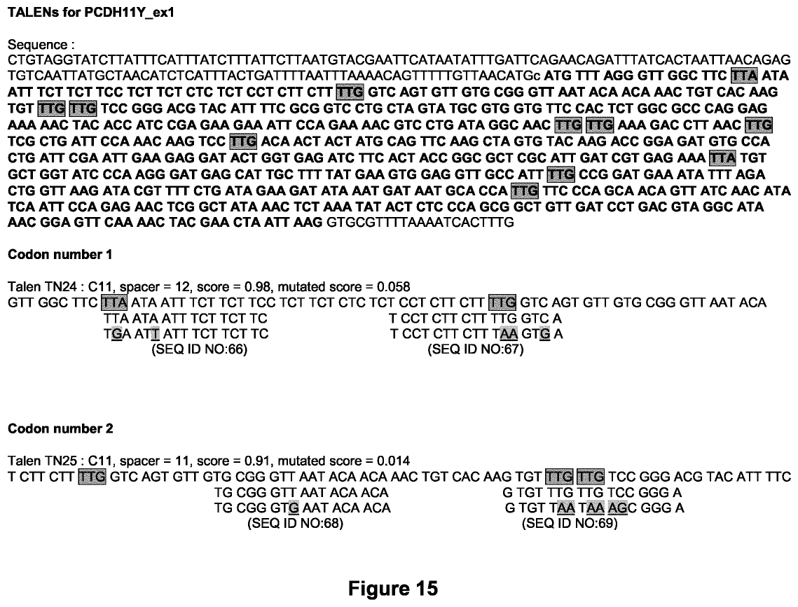

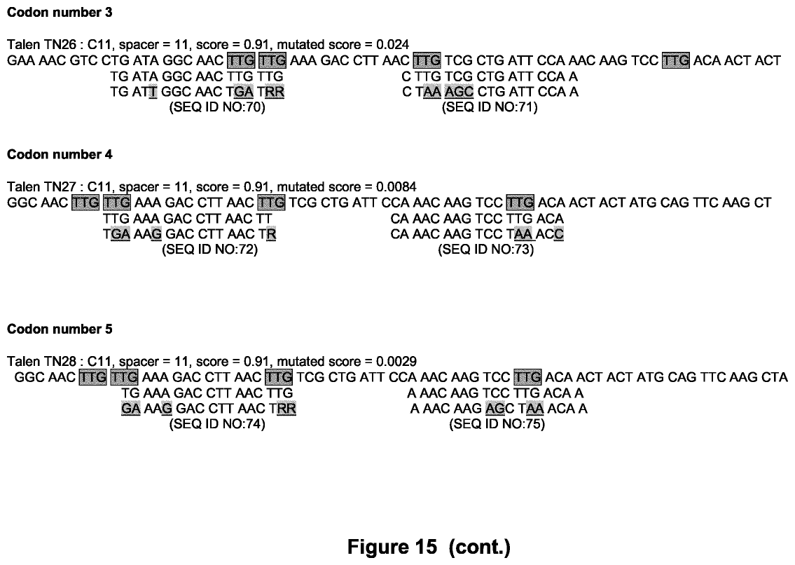

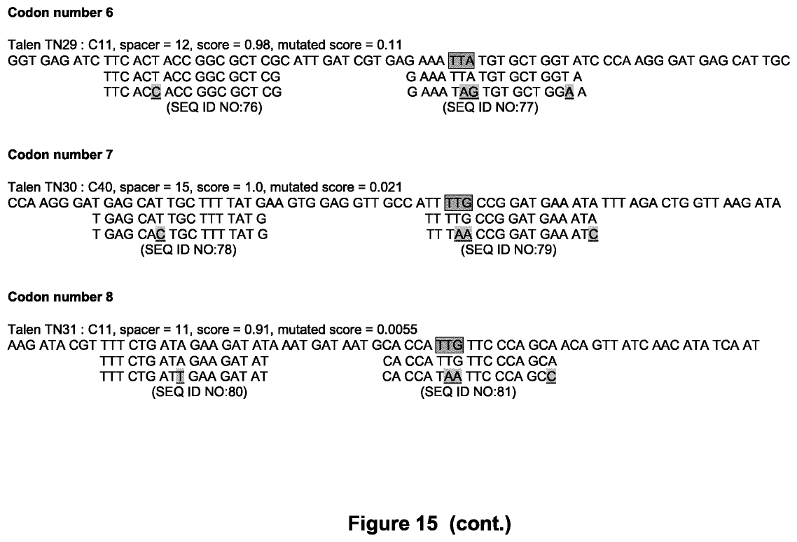

The strategy followed for substituting selected codon by stop codon is detailed in FIGS. 10 to 12 (without optimization) and FIGS. 13 to 15 (with optimization by using further substitutions involving synonymous codons).

Briefly, for each codon to be replaced by a stop codon, TALEN® were designed which have a binding site overlapping this codon, and looked for the one which was expected to be the most efficient. Then, it was looked at all the possibilities of stop codons one could introduce, and stop codons that decreased the most the score of recognition of the mutated exon by the TALEN were retained. Optionally, it is possible to mutate the exon sequence at the binding site to decrease even more the possibility that it will be cut by the TALEN. For obtaining such an optimization, it was looked at all codons overlapping these binding sites and searched for alternative synonymous codons that would introduce mutations to a frequency in the genome of interest higher than the one for the initial codon (to avoid to change codons to more unusual codons). The impact of the mutated codon on TALEN efficiency was examined and nucleotide triplet were selected that decreases the most the score. Single additional codon change was allowed in each TALEN half-binding site, and the codon change that has the greatest overall impact on TALEN efficiency was retained.

As for the scoring useful to select the appropriate TALE target sequences, each TALEN was scored:

against its native target (wild type sequence) to know its efficiency.

against the mutated target bearing the stop codon to know its selectivity and ensure it has a low probability to recognize the mutated target.

Such scoring involved:

the spacer size relative to the scaffold used for the TALEN.

the nucleotides that are not the expected ones relative to the RVD at the corresponding position.

the nature of the nucleotide at position 0 (if it is not a T).

The above strategy resulted into the identification of 31 target sequences into genes PCDH11Y_ex1, SRY_ex1 and PCDH11Y_ex1 allowing substitution of TTG and TTA codons by stop codon TAA, TAG or TGA, for which allele specific TALE-nucleases have been designed.

Tables 2 and 3 recapitulate the identified target sequence and the RVD sequences of the corresponding TALE-nucleases generated for efficient one-way allele specific codon substitution at the PCDH11Y_ex1, SRY_ex1 and PCDH11Y_ex1 loci.

TABLE 2
TALE-nucleases designed for stop codon replacement
(without optimization as per FIGS. 10 to 12)

| Talen | Left half | SEQ ID | ID Right half | SEQ ID | RVD sequence | RVD sequence |
|---|---|---|---|---|---|---|
| Name | target sequence | NO: # | target sequence | NO: # | Left monomer | Right Monomer |
| TN1 | TATAATAATCCTTAGGC | 20 | TTTGGCAAAACAGGAAC | 21 | NI-NG-NI-NI-NG-NI-NI- | NG-NG-NN-NN-HD-NI-NI- |
| | | | | | NG-HD-HD-NG-NG-NI-NN- | NI-NI-HD-NI-NN-NN-NI- |
| | | | | | NN-NG# | NI-NG# |
| TN2 | TAGGCCTCGATGGGTGG | 22 | TCTAATTCCCCTTTTGG | 23 | NI-NN-NN-HD-HD-NG-HD- | HD-NG-NI-NI-NG-NG-HD- |
| | | | | | NN-NI-NG-NN-NN-NN-NG- | HD-HD-HD-NG-NG-NG-NG- |
| | | | | | NN-NG# | NN-NG# |
| TN3 | TTAGAAGCTGCTATTGA | 24 | TACATCCACACTCACCT | 25 | NG-NI-NN-NI-NI-NN-HD- | NI-HD-NI-NG-HD-HD-NI- |
| | | | | | NG-NN-HD-NG-NI-NG-NG- | HD-NI-HD-NG-HD-NI-HD- |
| | | | | | NN-NG# | HD-NG# |
| TN4 | TTTGACAATGCAATCAT | 26 | TTGAATACGCTTAACAT | 27 | NG-NG-NN-NI-HD-NI-NI- | NG-NN-NI-NI-NG-NI-HD- |
| | | | | | NG-NN-HD-NI-NI-NG-HD- | NN-HD-NG-NG-NI-NI-HD- |
| | | | | | NI-NG# | NI-NG# |
| TN5 | TTACAGGCCATGCACAG | 28 | TCGATACTTA7AATTCG | 29 | NG-NI-HD-NI-NN-NN-HD- | HD-NN-NI-NG-NI-HD-NG- |
| | | | | | HD-NI-NG-NN-HD-NI-HD- | NG-NI-NG-NI-NI-NG-NG- |
| | | | | | NI-NG# | HD-NG# |
| TN6 | TCGGAAGGCGAAGATGC | 30 | TGCGGGAAGCAAACTGC | 31 | HD-NN-NN-NI-NI-NN-NN- | NN-HD-NN-NN-NN-NI-NI- |
| | | | | | HD-NN-NI-NI-NN-NI-NG- | NN-HD-NI-NI-NI-HD-NG- |
| | | | | | NN-NG# | NN-NG# |
| TN7 | TCCCGCTTCGGTACTCT | 32 | TACAACCTGTTGTCCAG | 33 | HD-HD-HD-NN-HD-NG-NG- | NI-HD-NI-NI-HD-HD-NG- |
| | | | | | HD-NN-NN-NG-NI-HD-NG- | NN-NG-NG-NN-NG-HD-HD- |
| | | | | | HD-NG# | NI-NG# |
| TN6 | TAGGCCACTTACCGCCC | 34 | TCCCGTTGCTGCGGTGA | 35 | NI-NN-NN-HD-HD-NI-HD- | HD-HD-HD-NN-NG-NG-NN- |
| | | | | | NG-NG-NI-HD-HD-NN-HD- | HD-NG-NN-HD-NN-NN-NG- |
| | | | | | HD-NG# | NN-NG# |
| TN9 | TTAATAATTTCTTCTTC | 36 | TGACCAAAAGAAGAGGA | 37 | NG-NI-NI-NG-NI-NI-NG- | NN-NI-HD-HD-NI-NI-NI- |
| | | | | | NG-NG-HD-NG-NG-HD-NG- | NI-NN-NI-NI-NN-NI-NN- |
| | | | | | NG-NG# | NN-NG# |
| TN10 | TGCGGGTTAAFACAACA | 38 | TCCCGGACAACAAACAC | 39 | NN-HD-NN-NN-NN-NG-NG- | HD-HD-HD-NN-NN-NI-HD- |
| | | | | | NI-NI-NG-NI-HD-NI-NI- | NI-NI-ND-NI-NI-NI-HD- |
| | | | | | HD-NG# | NI-NG# |
| TN11 | TCCGAGAAGAAAITCCA | 40 | TTCAACAAGTTGCCTAT | 41 | HD-HD-NN-NI-NN-NI-NI- | NG-HD-NI-NI-HD-NI-NI- |
| | | | | | NK-NI-NI-NI-NG-NG-HD- | NN-NG-NG-NM-HD-HD-NG- |
| | | | | | HD-NG# | NI-NG# |
| TN12 | TGAAAGACCTTAACTTG | 42 | TTGTCAAGGACTTGTTT | 43 | NN-NI-NI-NI-NN-NI-HD- | NG-NN-NG-HD-NI-NI-NN- |
| | | | | | HD-NG-NG-NI-NI-HD-NG- | NN-NI-HD-NG-NG-NN-NG- |
| | | | | | NG-NG# | NG-NG# |
| TN13 | TTCACTACCGGCGCTCG | 44 | TACCAGCACATAATTTC | 45 | NG-HD-NI-HD-NG-NI-HD- | NI-HD-HD-NI-NN-HD-NI- |
| | | | | | HD-NN-NN-HD-NN-HD-NG- | HD-NI-NG-NI-NI-NG-NG- |
| | | | | | HD-NG# | NG-NG# |
| TN14 | TGAGCATTGCTTTTATG | 46 | TTCATCCGGCAAAATGG | 47 | NN-NI-NN-HD-NI-NG-NG- | NG-HD-NI-NG-HD-HD-NN- |
| | | | | | NN-HD-NG-NG-NG-NG-NI- | NN-HD-NI-NI-NI-NI-NG- |
| | | | | | NG-NG# | NN-NG# |
| TN15 | TTCTGATAGAAGATATA | 48 | TTGCTGGGAACAATGGT | 49 | NG-HD-NG-NN-NI-NG-NI- | NG-NM-HD-NG-NN-NN-NN- |
| | | | | | NK-NI-NI-NN-NI-NG-NI- | NI-NI-HD-NI-NI-NG-NN- |
| | | | | | NG-NG# | NN-NG# |

TABLE 3
TALE-nucleases designed for stop codon replacement
(with optimization as per FIGS. 13 to 15)

| Talen | Left half | SEQ ID | Right half | SEQ ID | RVD sequence | RVD sequence |
|---|---|---|---|---|---|---|
| Name | target sequence | NO:# | target sequence | NO: # | Left monomer | Right Monomer |
| TN16 | TTTATAATAATCCTTAG | 50 | TGGCAAAACAGGAACCA | 51 | NG-NG-NI-NG-NI-NI-NG- | NN-NN-HD-NI-NI-NI-NI- |
| | | | | | NI-NI-NG-HD-HD-NG-NG- | HD-NI-NN-NN-NI-NI-HD- |
| | | | | | NI-NG# | HD-NG# |
| TN17 | TAGGCCTCGATGGGTGG | 52 | TTCTAATTCCCCTTTTG | 53 | NI-NN-NN-HD-HD-NG-HD- | NG-HD-NG-NI-NI-NG-NG- |
| | | | | | NN-NI-NG-NN-NN-NN-NG- | HD-HD-HD-HD-NG-NG-NG- |
| | | | | | NN-NG# | NG-NG# |
| TN18 | TTAGAAGCTGCTATTGA | 54 | TACATCCACACTCACCT | 55 | NG-NI-NN-NI-NI-NN-HD- | NI-HD-NI-NG-HD-HD-NI- |
| | | | | | NG-NN-HD-NG-NI-NG-NG- | HD-NI-HD-NG-HD-NI-HD- |
| | | | | | NN-NG# | HD-NG# |
| TN19 | TGCTTCTGCTATGTTAA | 56 | TGGACTGTAATCATCGC | 57 | NN-HD-NG-NG-HD-NG-NN- | NN-NN-NI-HD-NG-NN-NG- |
| | | | | | HD-NG-NI-NG-NN-NG-NG- | NI-NI-NG-HD-NI-NG-HD- |
| | | | | | NI-NG# | NN-NG# |
| TN20 | TTACAGGCCATGCACAG | 58 | TCGATACTTATAATTCG | 59 | NG-NI-HD-NI-NN-NN-HD- | HD-NN-NI-NG-NI-HD-NG- |
| | | | | | HD-NI-NG-NN-HD-NI-HD- | NG-NI-NG-NI-NI-NG-NG- |
| | | | | | NI-NG# | HD-NG# |
| TN21 | TCGGAAGGCGAAGATGC | 60 | TGCGGGAAGCAAACTGC | 61 | HD-NN-NN-NI-NI-NN-NN- | NN-HD-NN-NN-NN-NI-NI- |
| | | | | | HD-NN-NI-NI-NN-NI-NG- | NN-HD-NI-NI-NI-HD-NG- |
| | | | | | NN-NG# | NN-NG# |
| TN22 | TCCCGCTTCGGTACTCT | 62 | TACAACCTGTTGTCCAG | 63 | HD-HD-HD-NN-HD-NG-NG- | NI-HD-NI-NI-HD-HD-NG- |
| | | | | | HD-NN-NN-NG-NI-HD-NG- | NN-NG-NG-NN-NG-HD-HD- |
| | | | | | HD-NG# | NI-NG# |
| TN23 | TTACCGCCCATCAACGC | 64 | TGTAGCGGTCCCGTTGC | 65 | NG-NI-HD-HD-NN-HD-HD- | NN-NG-NI-NN-HD-NN-NN- |
| | | | | | HD-NI-NG-HD-NI-NI-HD- | NG-HD-HD-HD-NN-NG-NG- |
| | | | | | NN-NG# | NN-NG# |
| TN24 | TTAATAATTTCTTCTTC | 66 | TGACCAAAAGAAGAGGA | 67 | NG-NI-NI-NG-NI-NI-NG- | NN-NI-HD-HD-NI-NI-NI- |
| | | | | | NG-NG-HD-NG-NG-HD-NG- | NI-NN-NI-NI-NN-NI-NN- |
| | | | | | NG-NG# | NN-NG# |
| TN25 | TGCGGGTTAATACAACA | 68 | TCCCGGACAACAAACAC | 69 | NN-HD-NN-NN-NN-NG-NG- | HD-HD-HD-NN-NN-NI-HD- |
| | | | | | NI-NI-NG-NI-HD-NI-NI- | NI-NI-HD-NI-NI-NI-HD- |
| | | | | | HD-NG# | NI-NG# |
| TN26 | TGATAGGCAACTTGTTG | 70 | TTGGAATCAGCGACAAG | 71 | NN-NI-NG-NI-NN-NN-HD- | NG-NN-NN-NI-NI-NG-HD- |
| | | | | | NI-NI-HD-NG-NG-NN-NG- | NI-NN-HD-NN-NI-HD-NI- |
| | | | | | NG-NG# | NI-NG# |
| TN27 | TTGAAAGACCTTAACTT | 72 | TGTCAAGGACTTGTTTG | 73 | NG-NN-NI-NI-NI-NN-NI- | NN-NG-HD-NI-NI-NN-NN- |
| | | | | | HD-HD-NG-NG-NI-NI-HD- | NI-HD-NG-NG-NN-NG-NG- |
| | | | | | NG-NG# | NG-NG# |
| TN28 | TGAAAGACCTTAACTTG | 74 | TTGTCAAGGACTTGTTT | 75 | NN-NI-NI-NI-NN-NI-HD- | NG-NN-NG-HD-NI-NI-NN- |
| | | | | | HD-NG-NG-NI-NI-HD-NG- | NN-NI-HD-NG-NG-NN-NG- |
| | | | | | NG-NG# | NG-NG# |
| TN29 | TTCACTACCGGCGCTCG | 76 | TACCAGCACATAATTTC | 77 | NG-HD-NI-HD-NG-NI-HD- | NI-HD-HD-NI-NN-HD-NI- |
| | | | | | HD-NN-NN-HD-NN-HD-NG- | HD-NI-NG-NI-NI-NG-NG- |
| | | | | | HD-NG# | NG-NG# |
| TN30 | TGAGCATTGCTTTTATG | 78 | TATTTCATCCGGCAAAA | 79 | NN-NI-NN-HD-NI-NG-NG- | NI-NG-NG-NG-HD-NI-NG- |
| | | | | | NN-HD-NG-NG-NG-NG-NI- | HD-HD-NN-NN-HD-NI-NI- |
| | | | | | NG-NG# | NI-NG# |
| TN31 | TTTCTGATAGAAGATAT | 80 | TGCTGGGAACAATGGTG | 81 | NG-NG-HD-NG-NN-NI-NG- | NN-HD-NG-NN-NN-NN-NI- |
| | | | | | NI-NN-NI-NI-NN-NI-NG- | NI-HD-NI-NI-NG-NN-NN- |
| | | | | | NI-NG# | NG-NG# |

1-65. (canceled)

66. A method for allele-specific codon modification at the HBB locus in a cell, said method comprising at least:
a) introducing into a cell a TALE-nuclease or Mega-TALE targeting the E6V allele of hemoglobin B (HBB), said nuclease binding the genomic target sequence TGGAGAAGTC TGCCGTTACT GCCCTGTGGG GCAAGGTGAA CGTGGA (SEQ ID NO:14);
b) introducing into said cell a polynucleotide template comprising the sequence AGGAGAAGTC TGCCGTTACT GCCCTGTGGG GCAAGGTGAA CGTGGA (SEQ ID NO:13), abrogating cleavage of the polynucleotide template by said TALE-nuclease or mega-TALE;
c) cleaving said allele of HBB with said TALE-nuclease or mega-TALE in said cell; and
d) integrating said polynucleotide template at said HBB locus.

67. The method according to claim 66, wherein said cell is a stem cell or a blood cell.

68. The method according to claim 66, wherein said polynucleotide template further comprises at least one synonymous codon in the target sequence.

69. The method according to claim 68, wherein said polynucleotide template comprises 2 to 5 synonymous codons.

70. The method according to claim 66, wherein said polynucleotide template is in an AAV vector.

71. The method according to claim 66, wherein said TALE-nuclease or Mega-TALE comprises the RVD sequence: NN-NN-NI-NN-NI-NI-NN-NG-HD-NG-NN-HD-HD-NN-NG-NG.

72. The method according to claim 71, wherein said endonuclease is the TALE-nuclease HBB-E6V.

73. An engineered cell produced by the method of claim 68.

74. An engineered cell comprising:
a) a polynucleotide encoding a TALE-nuclease or Mega-TALE targeting the E6V allele of hemoglobin B (HBB), said nuclease binding the genomic target sequence TGGAGAAGTC TGCCGTTACT GCCCTGTGGG GCAAGGTGAA CGTGGA (SEQ ID NO:14) ; and
b) a polynucleotide template comprising the sequence AGGAGAAGTC TGCCGTTACT GCCCTGTGGG GCAAGGTGAA CGTGGA (SEQ ID NO:13).

75. The engineered cell according to claim 74, wherein said polynucleotide template further comprises at least one synonymous codon in the target sequence.

76. The engineered cell according to claim 74, wherein said polynucleotide template comprises 2 to 5 synonymous codons.

77. The engineered cell according to claim 74, wherein said TALE-nuclease or Mega-TALE comprises the RVD sequence: NN-NN-NI-NN-NI-NI-NN-NG-HD-NG-NN-HD-HD-NN-NG-NG.

78. The engineered cell according to claim 77, wherein said TALE-nuclease is the TALE-nuclease HBB-E6V.

79. The engineered cell according to claim 74, wherein said polynucleotide template is in a AAV vector.

80. The engineered cell according to claim 74, wherein said cell is a stem cell or a blood cell.

81. A kit for allele-specific codon modification at a HBB locus in a cell, said kit comprising at least:
a) polynucleotide encoding a TALE-nuclease or Mega-TALE targeting the E6V allele of hemoglobin B (HBB), said nuclease binding the genomic target sequence TGGAGAAGTC TGCCGTTACT GCCCTGTGGG GCAAGGTGAA CGTGGA (SEQ ID NO:14) ; and
b) a polynucleotide template comprising the sequence AGGAGAAGTC TGCCGTTACT GCCCTGTGGG GCAAGGTGAA CGTGGA (SEQ ID NO:13).

82. The kit according to claim 81, wherein said polynucleotide template is in an AAV vector.

83. The kit according to claim 81, wherein said TALE-nuclease or Mega-TALE comprises the RVD sequence: NN-NN-NI-NN-NI-NI-NN-NG-HD-NG-NN-HD-HD-NN-NG-NG.

84. The kit according to claim 83, wherein said TALE-nuclease is the TALE-nuclease HBV-E6V.

85. A TALE-nuclease or Mega-TALE, which selectively binds the target sequence:
(SEQ ID NO: 11)
5′-(T₀)GGAGAAGTCTGCCGTT.

86. The TALE nuclease of claim 85, comprising the RVD sequence: NN-NN-NI-NN-NI-NI-NN-NG-HD-NG-NN-HD-HD-NN-NG-NG.

87. The TALE-nuclease of claim 86, which comprises HBB-E6V-L1.
